(19) United States
(12) Patent Application Publication
(10) Pub. No.: US 2004/0229927 A1
(43) Pub. Date: Nov. 18, 2004

(54) Imidazole derivatives for treatment of allergic and hyperproliferative disorders

(76) Inventors: Jagadish C. Sircar, San Diego, CA (US); Richard J. Thomas, San Diego, CA (US); Mark L. Richards, San Diego, CA (US); Haripada Khatuya, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/821,667

(22) Filed: Apr. 9, 2004

Related U.S. Application Data
(60) Provisional application No. 60/462,090, filed on Apr. 10, 2003.

(51) Int. Cl. A61K 31/4164

(52) U.S. Cl. 514/396

(57) ABSTRACT

The preferred embodiments are directed to small molecule inhibitors of the IgE response to allergens, which are useful in the treatment of allergy and/or asthma or any diseases where IgE is pathogenic. The preferred embodiments also relate to imidazole molecules that are cellular proliferation inhibitors and thus are useful as anticancer agents. The preferred embodiments further relate to small molecules which suppress cytokines and leukocytes.

### CROSS-REFERENCE TO RELATED APPLICATIONS

This application claims the benefit under 35 U.S.C. § 119(e) of U.S. Provisional Patent Application No. 60/462,090, filed Apr. 10, 2003.

### BACKGROUND OF THE INVENTION

1. Field of the Invention

This invention relates to small molecule inhibitors of the IgE response to allergens that are useful in the treatment of allergy and/or asthma or any diseases where IgE is pathogenic. This invention also relates to small molecules that are proliferation inhibitors and thus they are useful as anticancer agents. This invention further relates to small molecules which suppress cytokines and leukocytes.

2. Description of the Related Art

Allergies and Asthma

An estimated 10 million persons in the United States have asthma, about 5% of the population. The estimated cost of asthma in the United States exceeds $6 billion. About 25% of patients with asthma who seek emergency care require hospitalization, and the largest single direct medical expenditure for asthma has been inpatient hospital services (emergency care), at a cost of greater than $1.6 billion. The cost for prescription medications, which increased 54% between 1985 and 1990, was close behind at $1.1 billion (Kelly, Pharmacotherapy 12:13S-21S (1997)).

According to the National Ambulatory Medical Care Survey, asthma accounts for 1% of all ambulatory care visits, and the disease continues to be a significant cause of missed school days in children. Despite improved understanding of the disease process and better drugs, asthma morbidity and mortality continue to rise in this country and worldwide (U.S. Department of Health and Human Services; 1991, publication no. 91-3042). Thus, asthma constitutes a significant public health problem.

The pathophysiologic processes that attend the onset of an asthmatic episode can be broken down into essentially two phases, both marked by bronchoconstriction, that causes wheezing, chest tightness, and dyspnea. The first, early phase asthmatic response is triggered by allergens, irritants, or exercise. Allergens cross-link immunoglobulin E (IgE) molecules bound to receptors on mast cells, causing them to release a number of pre-formed inflammatory mediators, including histamine. Additional triggers include the osmotic changes in airway tissues following exercise or the inhalation of cold, dry air. The second, late phase response that follows is characterized by infiltration of activated eosinophils and other inflammatory cells into airway tissues, epithelial desquamonon, and by the presence of highly viscous mucus within the airways. The damage caused by this inflammatory response leaves the airways “primed” or sensitized, such that smaller triggers are required to elicit subsequent asthma symptoms.

A number of drugs are available for the palliative treatment of asthma; however, their efficacies vary markedly. Short-acting β₂-adrenergic agonists, terbutaline and albuterol, long the mainstay of asthma treatment, act primarily during the early phase as bronchodilators. The newer long-acting β₂-agonists, salmeterol and formoterol, may reduce the bronchoconstrictive component of the late response. However, because the β₂-agonists do not possess significant antiinflammatory activity, they have no effect on bronchial hyperreactivity.

Numerous other drugs target specific aspects of the early or late asthmatic responses. For example, antihistamines, like loratadine, inhibit early histamine-mediated inflammatory responses. Some of the newer antihistamines, such as azelastine and ketotifen, may have both antiinflammatory and weak bronchodilatory effects, but they currently do not have any established efficacy in asthma treatment. Phosphodiesterase inhibitors, like theophylline/xanthines, may attenuate late inflammatory responses, but there is no evidence that these compounds decrease bronchial hyperreactivity. Anticholinergics, like ipratopium bromide, which are used in cases of acute asthma to inhibit severe bronchoconstriction, have no effect on early or late phase inflammation, no effect on bronchial hyperreactivity, and therefore, essentially no role in chronic therapy.

The corticosteroid drugs, like budesonide, are the most potent antiinflammatory agents. Inflammatory mediator release inhibitors, like cromolyn and nedocromil, act by stabilizing mast cells and thereby inhibiting the late phase inflammatory response to allergen. Thus, cromolyn and nedocromil, as well as the corticosteroids, all reduce bronchial hyperreactivity by minimizing the sensitizing effect of inflammatory damage to the airways. Unfortunately, these antiinflammatory agents do not produce bronchodilation.

Several new agents have been developed that inhibit specific aspects of asthmatic inflammation. For instance, leukotriene receptor antagonists (ICI-204, 219, accolate), specifically inhibit leukotriene-mediated actions. The leukotrienes have been implicated in the production of both airway inflammation and bronchoconstriction.

Thus, while numerous drugs are currently available for the treatment of asthma, these compounds are primarily palliative and/or have significant side effects. Consequently, new therapeutic approaches which target the underlying cause rather than the cascade of symptoms would be highly desirable. Asthma and allergy share a common dependence on IgE-mediated events. Indeed, it is known that excess IgE production is the underlying cause of allergies in general and allergic asthma in particular (Duplantier and Cheng, Ann. Rep. Med. Chem. 29:73-81 (1994)). Thus, compounds that lower IgE levels may be effective in treating the underlying cause of asthma and allergy.

None of the current therapies eliminate the excess circulating IgE. The hypothesis that lowering plasma IgE may reduce the allergic response, was confirmed by recent clinical results with chimeric anti-IgE antibody, CGP-51901, and recombinant humanized monoclonal antibody, rhuMAB-E25. Indeed, three companies, Tanox Biosystems, Inc., Genentech Inc. and Novartis AG are collaborating in the development of a humanized anti-IgE antibody (BioWorld® Today, Feb. 26, 1997, p. 2) which will treat allergy and asthma by neutralizing excess IgE. Tanox has already successfully tested the anti-IgE antibody, CGP-51901, which reduced the severity and duration of nasal symptoms of allergic rhinitis in a 155-patient Phase II trial (Scrip #2080, Nov. 24, 1995, p.26). Genentech recently disclosed positive results from a 536 patient phase-II/III trials of its recombinant humanized monoclonal antibody, rhuMAB-E25 (BioWorld® Today, Nov. 10, 1998, p. 1). The antibody, rhuMAB-E25, administered by injection (highest dose 300 mg every 2 to 4 weeks as needed) provided a 50% reduction in the number of days a patient required additional “rescue” medicines (antihistimines and decongestants), compared to placebo. More recently, Dr. Henry Milgrom et al. of the National Jewish Medical and Research Center in Denver, Colo., published the clinical results of rhuMAB-25 in moderate to severe asthma patients (317 patients for 12 weeks, iv injection every two weeks) and concluded that this drug is “going to be a breakthrough” (New England Journal of Medicine, Dec. 23, 1999). A Biologics License Application (BLA) for this product has been submitted to the FDA in June, 2000 jointly by Novartis Pharmaceuticals Corporation, Tanox Inc., and Genentech, Inc. The positive results from anti-IgE antibody trials suggest that therapeutic strategies aimed at IgE down-regulation may be effective.

Cancer and Hyperproliferation Disorders

Cellular proliferation is a normal process that is vital to the normal functioning of most biological processes. Cellular proliferation occurs in all living organisms and involves two main processes: nuclear division (mitosis), and cytoplasmic division (cytokinesis). Because organisms are continually growing and replacing cells, cellular proliferation is essential to the vitality of the healthy cell. The disruption of normal cellular proliferation can result in a variety of disorders. For example, hyperproliferation of cells may cause psoriasis, thrombosis, atherosclerosis, coronary heart disease, myocardial infarction, stroke, smooth muscle neoplasms, uterine fibroid or fibroma, and obliterative diseases of vascular grafts and transplanted organs. Abnormal cell proliferation is most commonly associated with tumor formation and cancer.

Cancer is a major disease and is one of the leading causes of mortality both in the United States and internationally. Indeed, cancer is the second leading cause of death in the United States. According to the National Institute of Health, the overall annual cost for cancer is approximately $107 billion, which includes $37 billion for direct medical costs, $11 billion for indirect costs of lost productivity due to illness and $59 billion for indirect costs of lost productivity due to premature death. Not surprisingly, considerable efforts are underway to develop new treatments and preventative measures to combat this devastating illness.

Currently, cancer is primarily treated using a combination of surgery, radiation and chemotherapy. Chemotherapy involves the use of chemical agents to disrupt the replication and metabolism of cancerous cells. Chemotherapeutic agents which are currently being used to treat cancer can be classified into five main groups: natural products and their derivatives; anthacyclines; alkylating agents; antiproliferatives and hormonal agents.

### SUMMARY OF THE INVENTION

It is one object of embodiments to provide imidazole compounds and methods thereof to modulate IgE. It is another object to provide imidazole compositions and methods to inhibit cell proliferation. It is yet another object of embodiments to inhibit cytokines and leukocytes, including but not limited to IL-4, IL-5, eosinophils and lymphocytes.

One family of small molecules of several embodiments is defined by the following genus (Genus 1):

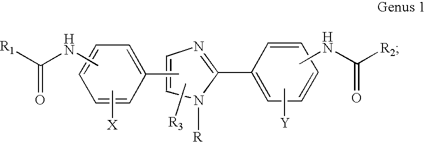

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R¹ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

One family of small molecule IgE inhibitors of the preferred embodiments is defined by the following genus (Genus 2):

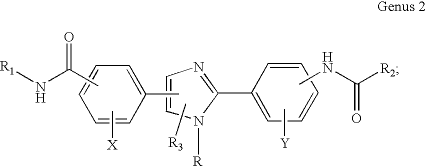

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylarninoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

One family of small molecule IgE inhibitors of the preferred embodiments is defined by the following genus (Genus 3):

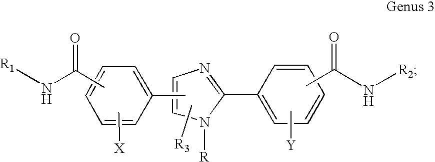

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

One family of small molecule IgE inhibitors of the preferred embodiments is defined by the following genus (Genus 4):

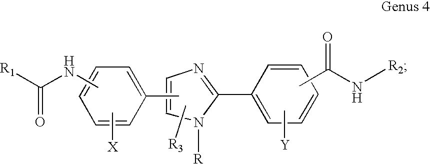

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

For each chemical structure disclosed herein, the hydrogen atoms on the heteroatoms may have been omitted for clarity purposes. Where open valences on heteroatoms are indicated, it is assumed that these valences are filled by hydrogen atoms.

It is assumed that the imidazole compounds are present in either of the tautomeric forms or mixture thereof.

A method for treating a disease condition associated with excess IgE and/or abnormal cell proliferation (i.e. cancer) in a mammal is also disclosed. In one aspect, the method comprises the step of administering to the mammal an IgE-suppressing amount or anti-cell proliferation amount of a pharmaceutical formulation comprising at least one imidazole compound from the above-disclosed small molecule families.

In accordance with a variation of the method of treatment, the small molecule IgE-suppressing compound may be administered in conjunction with at least one additional agent, which is active in reducing a symptom associated with an allergic reaction. In one embodiment, the small molecule inhibitor may be mixed with at least one additional active ingredient to form a pharmaceutical composition. Alternatively, the small molecule inhibitor may be co-administered at the same time or according to different treatment regimens with the at least one additional active agent.

The at least one additional active ingredient may be a short-acting β₂-adrenergic agonist selected from the group consisting of terbutaline and albuterol; a long-acting β₂-adrenergic agonist selected from the group consisting of salmeterol and formoterol; an antihistamine selected from the group consisting of loratadine, azelastine and ketotifen; a phosphodiesterase inhibitor, an anticholinergic agent, a corticosteroid, an inflammatory mediator release inhibitor or a leukotriene receptor antagonist.

In another embodiment, the imidazole compound may be administered in conjunction with at least one additional active agent. These active agents include antifungals, antivirals, antibiotics, anti-inflammatories, and anticancer agents. Anticancer agents include, but are not limited to, alkylating agents (lomustine, carmustine, streptozocin, mechlorethamine, melphalan, uracil nitrogen mustard, chlorambucil cyclophosphamide, iphosphamide, cisplatin, carboplatin mitomycin thiotepa dacarbazine procarbazine, hexamethyl melamine, triethylene melamine, busulfan, pipobroman, and mitotane); antimetabolites (methotrexate, trimetrexate pentostatin, cytarabine, ara-CMP, fludarabine phosphate, hydroxyurea, fluorouracil, floxuridine, chlorodeoxyadenosine, gemcitabine, thioguanine, and 6-mercaptopurine); DNA cutters (bleomycin); topoisomerase I poisons (topotecan irinotecan and camptothecin); topoisomerase II poisons (daunorubicin, doxorubicin, idarubicin, mitoxantrone, teniposide, and etoposide); DNA binders (dactinomycin, and mithramycin); and spindle poisons (vinblastine, vincristine, navelbine, paclitaxel, and docetaxel).

In another embodiment, the imidazole compounds of the preferred embodiments are administered in conjunction with one or more other therapies. These therapies include, but are not limited to radiation, immunotherapy, gene therapy and surgery. These combination therapies may be administered simultaneously or sequentially. For example, radiation may be administered along with the administration of imidazole compounds, or may be administered at any time before or after administration of imidazole compounds.

A dose of about 0.01 mg to about 100 mg per kg body weight per day of the small molecule IgE inhibitory compound is preferably administered in divided doses daily.

A method for treating a disease condition associated with excess IgE or abnormal cell proliferation in a mammal is also disclosed which comprises the step of administering to the mammal an therapeutic amount of a pharmaceutical formulation comprising at least one compound selected from Genera 1-4.

The methods provided herein for treating diseases and processes mediated by undesired, uncontrolled or abnormal cell proliferation, such as cancer, involve administering to a mammal a composition of the imidazole compounds disclosed herein to inhibit cell proliferation. The method is particularly useful for preventing or treating tumor formation and progression. In the preferred embodiments, the compounds and methods disclosed are especially useful in treating estrogen receptor positive and estrogen receptor negative type breast cancers.

Other variations within the scope of the present invention may be more fully understood with reference to the following detailed description.

### DETAILED DESCRIPTION OF THE PREFERRED EMBODIMENT

The preferred embodiments are directed to small molecule inhibitors of IgE which are useful in the treatment of allergy and/or asthma or any diseases where IgE is pathogenic. The inhibitors may affect the synthesis, activity, release, metabolism, degradation, clearance and/or pharmacokinetics of IgE. The particular compounds disclosed herein were identified by their ability to suppress IgE levels in both ex vivo and in vivo assays. The compounds disclosed in the preferred embodiments are also useful in the treatment of diseases associated with abnormal cellular proliferation, including, but not limited to, tumorgenesis and other proliferative diseases such as cancers, inflammatory disorders and circulatory diseases. Development and optimization of clinical treatment regimens can be monitored by those of skill in the art by reference to the ex vivo and in vivo assays described below. In addition, several embodiments are directed to imidazole compounds that inhibit cytokines and leukocytes, including but not limited to IL-4, IL-5, eosinophils and lymphocytes.

Ex Vivo Assay

This system begins with in vivo antigen priming and measures secondary antibody responses in vitro. The basic protocol was documented and optimized for a range of parameters including: antigen dose for priming and time span following priming, number of cells cultured in vitro, antigen concentrations for eliciting secondary IgE (and other Ig's) response in vitro, fetal bovine serum (FBS) batch that will permit optimal IgE response in vitro, the importance of primed CD4+ T cells and hapten-specific B cells, and specificity of the ELISA assay for IgE (Marcelletti and Katz, Cellular Immunology 135:471-489 (1991); incorporated herein by reference).

The actual protocol utilized for this project was adapted for a more high throughput analyses. BALB/cByj mice were immunized i.p. with 10 μg DNP-KLH adsorbed onto 4 mg alum and sacrificed after 15 days. Spleens were excised and homogenized in a tissue grinder, washed twice, and maintained in DMEM supplemented with 10% FBS, 100 U/ml penicillin, 100 μg/ml streptomycin and 0.0005% 2-mercaptoethanol. Spleen cell cultures were established (2-3 million cells/ml, 0.2 ml/well in quadruplicate, 96-well plates) in the presence or absence of DNP-KLH (10 ng/ml). Test compounds (2 μg/ml and 50 ng/ml) were added to the spleen cell cultures containing antigen and incubated at 37° C. for 8 days in an atmosphere of 10% CO₂.

Culture supernatants were collected after 8 days and Ig's were measured by a modification of the specific isotype-selective ELISA assay described by Marcelletti and Katz (supra). The assay was modified to facilitate high throughput. ELISA plates were prepared by coating with DNP-KLH or DNP-OVA overnight. After blocking with bovine serum albumin (BSA), an aliquot of each culture supernatant was diluted (1:4 in phosphate buffered saline (PBS) with BSA, sodium azide and Tween 20), added to the ELISA plates, and incubated overnight in a humidified box at 4° C. IgE levels were quantitated following successive incubations with biotinylated-goat antimouse IgE (b-GAME), AP-streptavidin and substrate.

Antigen-specific IgG1 was measured similarly, except that culture supernatants were diluted 200-fold and biotinylated-goat antimouse IgG1 (b-GAMG1) was substituted for b-GAME. IgG2a was measured in ELISA plates that were coated with DNP-KLH following a 1:20 dilution of culture supernatants and incubation with biotinylated-goat antimouse IgG2a (b-GAMG2a). Quantitation of each isotype was determined by comparison to a standard curve. The level of detectability of all antibody was about 200-400 pg/ml and there was less than 0.001% cross-reactivity with any other Ig isotype in the ELISA for IgE.

In Vivo Assay

Compounds found to be active in the ex vivo assay (above) were further tested for their activity in suppressing IgE responses in vivo. Mice receiving low-dose radiation prior to immunization with a carrier exhibited an enhanced IgE response to challenge with antigen 7 days later. Administration of the test compounds immediately prior to and after antigen sensitization, measured the ability of that drug to suppress the IgE response. The levels of antigen specific IgE, IgG1 and IgG2a in serum were compared.

Female BALB/cByj mice were irradiated with 250 rads 7 hours after initiation of the daily light cycle. Two hours later, the mice were immunized i.p. with 2 μg of KLH in 4 mg alum. Two to seven consecutive days of drug injections were initiated 6 days later on either a once or twice daily basis. Typically, i.p. injections and oral gavages were administered as suspensions (150 μl/injection) in saline with 10% ethanol and 0.25% methylcellulose. Each treatment group was composed of 5-6 mice. On the second day of drug administration, 2 μg of DNP-KLH was administered i.p. in 4 mg alum, immediately following the morning injection of drug. Mice were bled 7-21 days following DNP-KLH challenge.

Antigen-specific IgE, IgG1 and IgG2a antibodies were measured by ELISA. Periorbital bleeds were centrifuged at 14,000 rpm for 10 min, the supernatants were diluted 5-fold in saline, and centrifuged again. Antibody concentrations of each bleed were determined by ELISA of four dilutions (in triplicate) and compared to a standard curve: anti-DNP IgE (1:100 to 1:800), anti-DNP IgG2a (1:100 to 1:800), and anti-DNP IgG1 (1:1600 to 1:12800).

### Active Compounds of Preferred Embodiments

The following series of compounds, identified under subheadings Genus 1-4 were found to be potent inhibitors of IgE in both ex-vivo and in vivo models. These compounds also exhibit anti-proliferative effects, and, as such, may be used as agents to treat hyperproliferation disorders, including cancer.

As used herein, alkyl refers to a straight chain, branched, or cyclic group of carbon atoms, including, but not limited to, methyl, ethyl, n-propyl, isopropyl, n-butyl, isobutyl, tert-butyl, n-hexyl, and the like.

As used herein, aryl refers to an aromatic carbocyclic group. Examples of aryl groups include, but are not limited to, phenyl, naphthyl and biphenyl.

As used herein, arylalkyl refers to an aryl-alkyl-group in which the aryl and alkyl portions are in accordance with the previous descriptions. Examples include, but are not limited to, benzyl, 1-phenethyl, 2-phenethyl, phenpropyl, phenbutyl, phenpentyl, and napthylmethyl.

As used herein, dialkylaminoalkyl refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

As used herein, halogen refers to fluoro, chloro, bromo, or iodo.

As used herein, alkoxy refers to an alkyl group, as defined above, having an oxygen attached thereto. Representative alkoxyl groups include, but are not limited to, methoxy, ethoxy, propyloxy, tert-butoxy, adamantyloxy, and the like.

As used herein, hydroxyalkyl refers to alkyl group that is substituted with at least one hydroxy group. Examples of hydroxyalkyl include, but are not limited to, hydroxymethyl, 2-hydroxyethyl, 3-hydroxypropyl, hydroxyadamantyl, and the like.

As used herein, cycloalkyl refers a cyclic form of alkyl group. Examples of cycloalkyl groups include, but are not limited to, cyclopropyl, cyclobutyl, cyclopentyl, cyclohexyl, cycloheptyl, and cyclooctyl.

As used herein, polycyclic aliphatic group refers to a substituted cycloalkyl group in which the substitution is at least one cycloalkyl group. The relationship of the substitution of one cycloalkyl group to the other can be isolated rings (no common atoms), spiro rings (one common atom), fused rings (one common bond), or bridged rings (two common atoms). Polycyclic aliphatic groups of fused rings type and bridged rings type include, but are not limited to, bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, adamantan-1-yl, adamantan-2-yl, and norbomyl.

As used herein, heterocyclic refers to a cyclic group having, as ring members, atoms of at least two different elements. Preferably, one of the elements is carbon. A heterocyclic group or ring can be saturated, unsaturated or heteroaromatic; unless defined otherwise, it preferably contains one or more, in particular 1, 2 or 3, heteroatoms in the heterocyclic ring, preferably from the group consisting of N, O and S. The heterocyclic group can, for example, be a heteroaromatic group or ring (heteroaryl), such as, for example, a mono-, bi- or polycyclic aromatic system in which at least 1 ring contains one or more heteroatoms. The terms heterocyclic and heterocyclyl may be used interchangeably herein.

As used herein, heteroaryl refers to a cyclic group that is a class of heterocyclyl group derived from heteroarenes by removal of a hydrogen atom from any ring atom. Heteroarenes are heterocyclic compounds formally derived from arenes by replacement of one or more methiine (—C═) and/or vinylene (—CH═CH—) groups by trivalent or divalent heteroatoms, respectively, in such a way as to maintain the continuous π-electron system characteristic of aromatic systems and a number of out of plane π-electrons corresponding to the Hückel rule (4 n+2). As used herein, the terms heteroaryl, hetaryl, heteroarene, hetarene, and heteroaromatic can be used interchangeably.

As noted above, a heteroaromatic group can be, for example, a mono-, bi- or polycyclic aromatic system in which at least 1 ring contains one or more heteroatoms. A heteroaromatic ring can contain one heteroatom from the group consisting of N, O and S, for example pyridyl, pyrrolyl, thienyl or furyl; furthermore, a heteroaromatic ring can contain 2 or 3 heteroatoms, for example pyrimidinyl, pyridazinyl, pyrazinyl, triazinyl, thiazolyl, thiadiazolyl, oxazolyl, isoxazolyl, pyrazolyl, imidazolyl and triazolyl.

As used herein, a substituted group is derived from the unsubstituted parent structure in which there has been an exchange of one or more hydrogen atoms for another atom or group.

Compounds of Genera 1-4 can exist in tautomeric forms by virtue of the imidazole ring: the N-hydrogen atom can tautomerize from one nitrogen atom to the other of that ring. All such isomers including diastereomers and enantiomers are covered by the embodiments. It is assumed that the imidazole compounds are present in either of the tautomeric forms or mixture thereof.

Compounds of Genus 1

One family of small molecule IgE inhibitors is defined by the following genus (Genus 1):

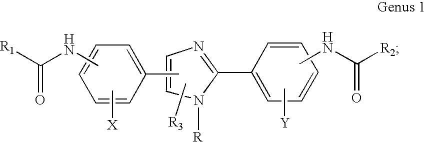

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

Compounds of Genus 1 may be synthesized by any conventional reactions known in the art. Examples of syntheses include the following reactions, designated Synthetic Schemes 1-8.

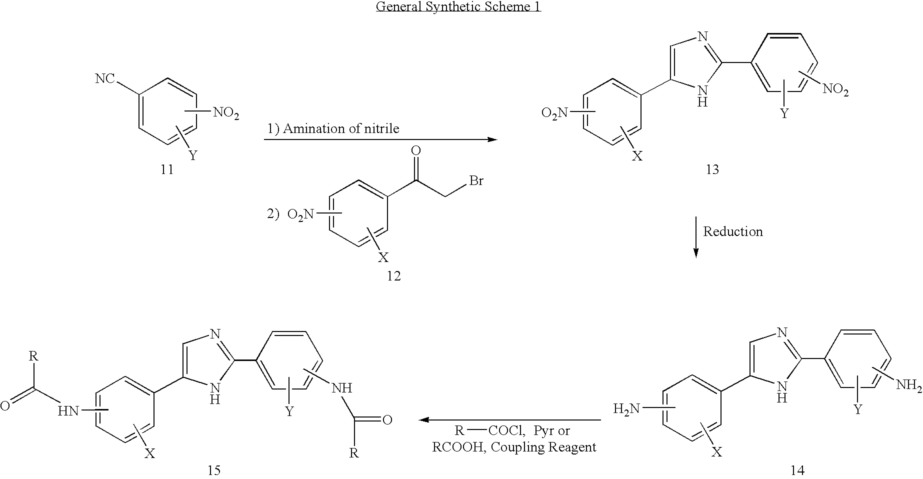

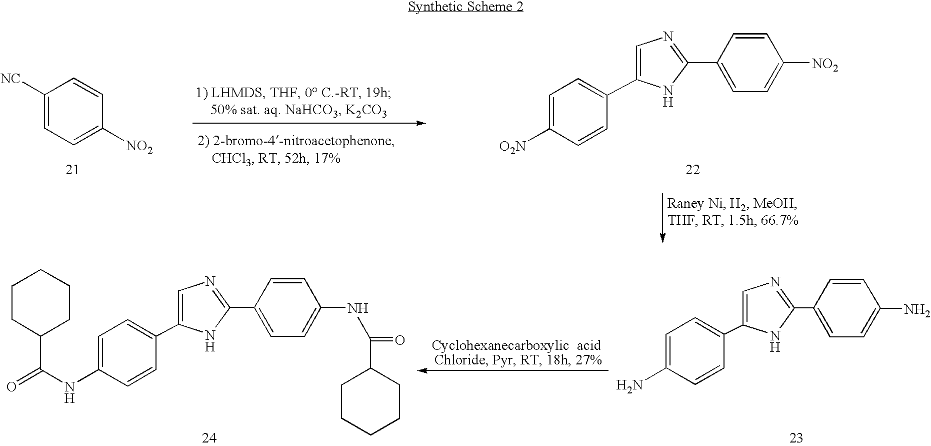

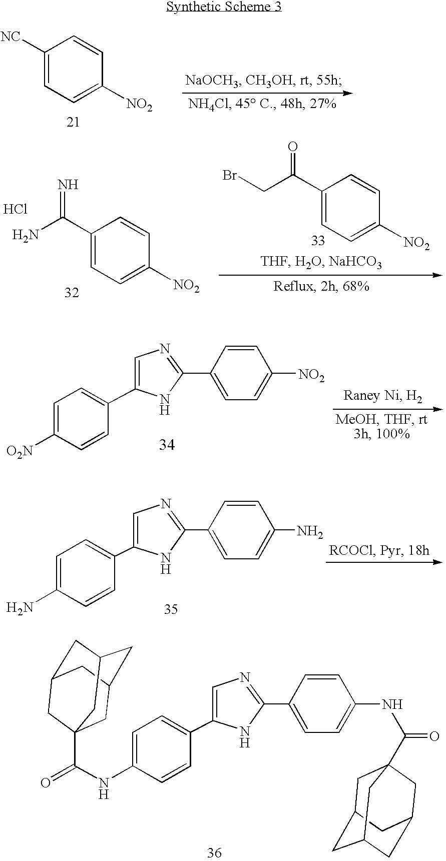

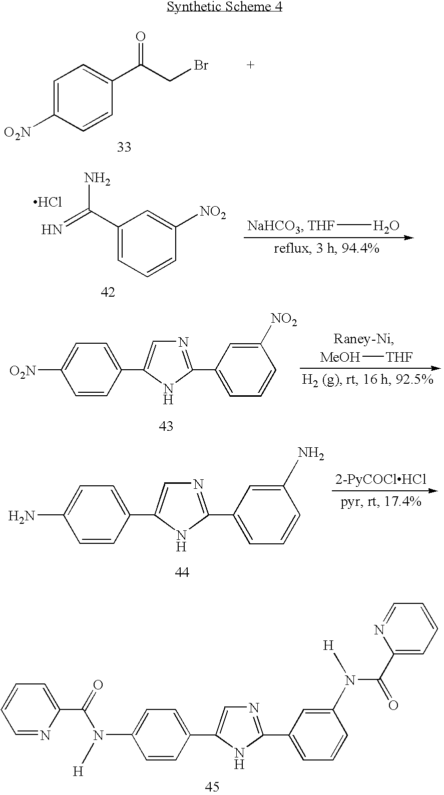

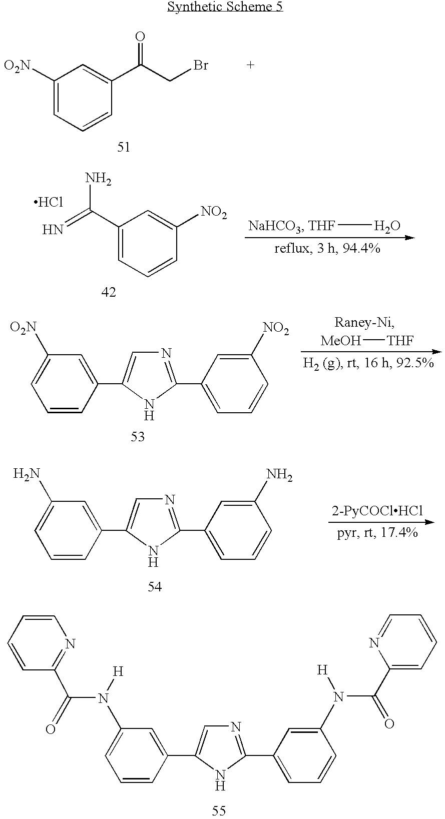

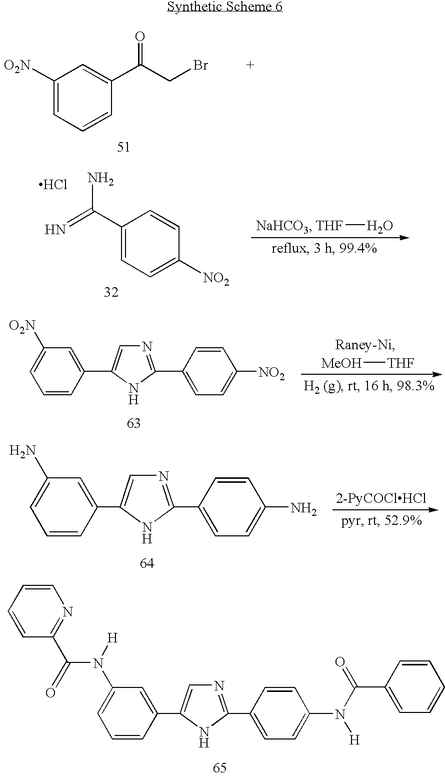

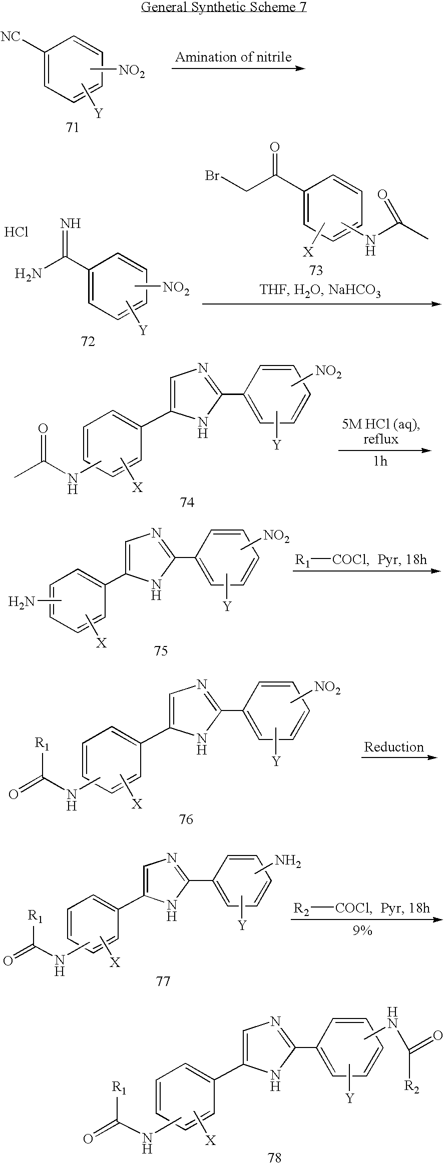

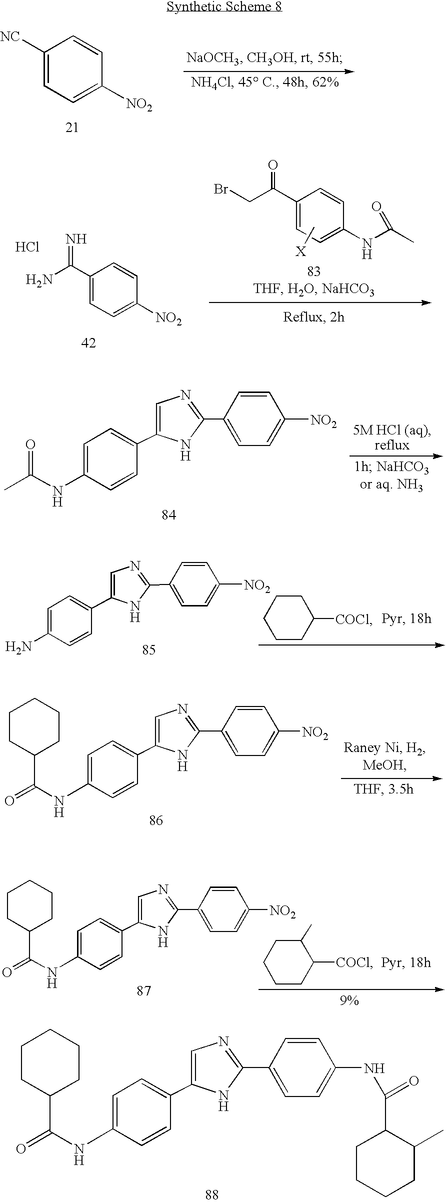

Accordingly, a preferred method of preparing a compound or salt thereof having the formula:

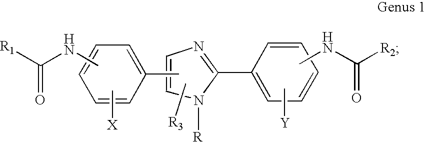

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl;

comprises the following steps: converting a Y-substituted-nitro-benzonitrile to a Y-substituted nitro-benzamidine; reacting the Y-substituted nitro-benzamidine with X-substituted nitro-phenacyl halide to form a species of the formula 13

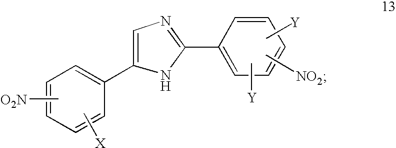

reducing the species of the formula 13 to form a species of the formula 14

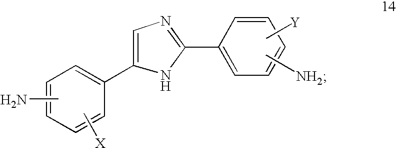

and acylating the species of the formula 14 to form a species of the formula 15

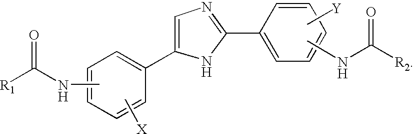

Accordingly, another preferred method of preparing

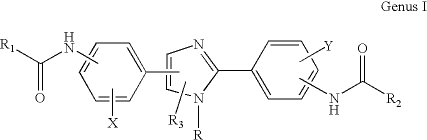

comprises the following steps: converting a Y-substituted nitro-benzonitrile to a Y-substituted nitro-benzamidine; reacting the Y-substituted nitro-benzamidine with X-substituted acetamido-phenacyl halide to form species of the formula 74

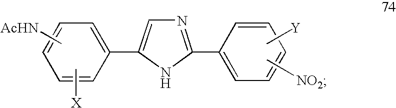

hydrolyzing the species of the formula 74 to form a species of the formula 75

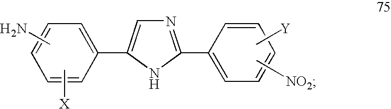

acylating the species of the formula 75 to from a species of the formula 76

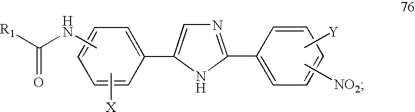

reducing the species of the formula 76 to form a species of the formula 77

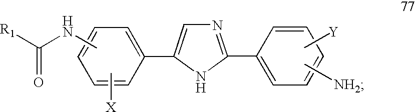

and acylating the species of the formula 77 to from a species of the formula 78

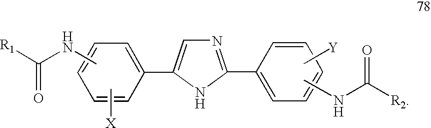

Synthesis of the Compounds of Genus 1

Synthetic Schemes 1-8 shows methods that can be used to prepare the compounds of Genus 1. One skilled in the art will appreciate that a number of different synthetic reaction schemes may be used to synthesize the compounds of Genus 1. Further, one skilled in the art will understand that a number of different solvents, coupling agents and reaction conditions can be used in the syntheses reactions to yield comparable results.

One skilled in the art will appreciate variations in the sequence and further, will recognize variations in the appropriate reaction conditions from the analogous reactions shown or otherwise known which may be appropriately used in the processes above to make the compounds of Synthetic Schemes 1-8.

In the processes described herein for the preparation of the compounds of Synthetic Schemes 1-8 of the preferred embodiments, the requirements for protective groups are generally well recognized by one skilled in the art of organic chemistry, and accordingly the use of appropriate protecting groups is necessarily implied by the processes of the schemes herein, although such groups may not be expressly illustrated. Introduction and removal of such suitable protecting groups are well known in the art of organic chemistry; see for example, T. W. Greene, “Protective Groups in Organic Synthesis”, Wiley (New York), 1981.

The products of the reactions described herein are isolated by conventional means such as extraction, distillation, chromatography, and the like.

Starting materials not described herein are available commercially, are known, or can be prepared by methods known in the art.

The salts of the compounds of Synthetic Schemes 1-8 described above are prepared by reacting the appropriate base or acid with a stoichiometric equivalent of the compounds of Synthetic Schemes 1-8.

Compounds of Genus 2

One family of small molecule IgE inhibitors is defined by the following genus (Genus 2):

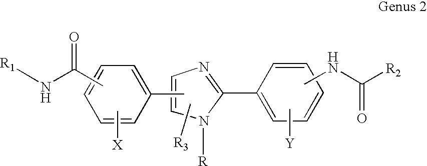

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

Compounds of Genus 2 may be synthesized by any conventional reactions known in the art. Examples of syntheses include the following reactions, designated Synthetic Schemes 9-13.

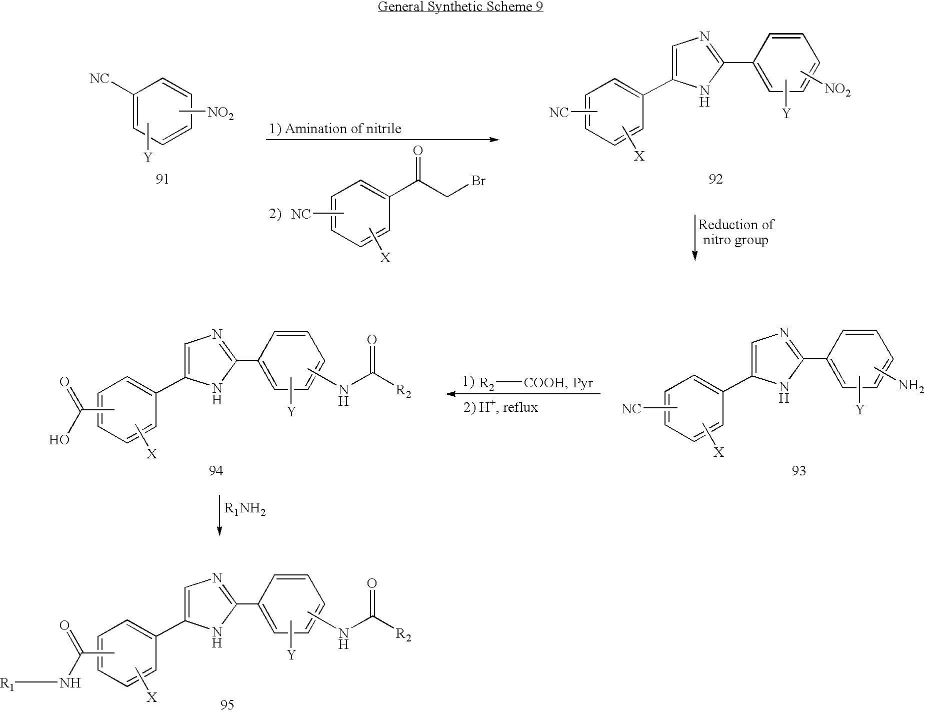

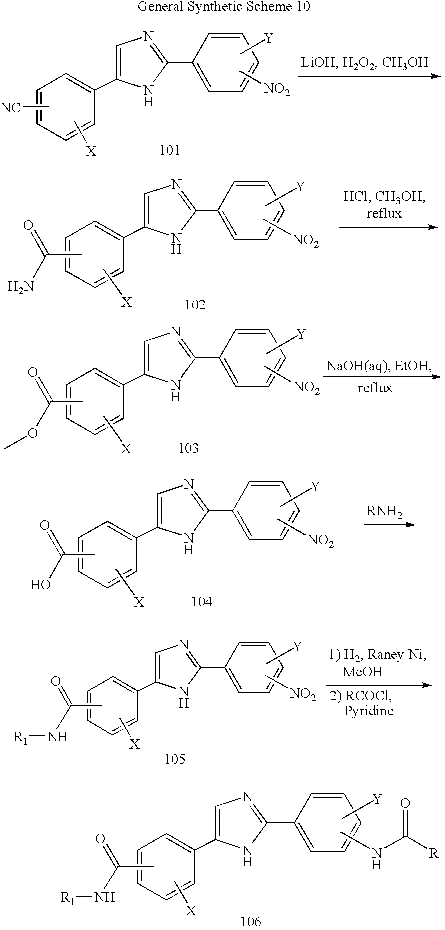

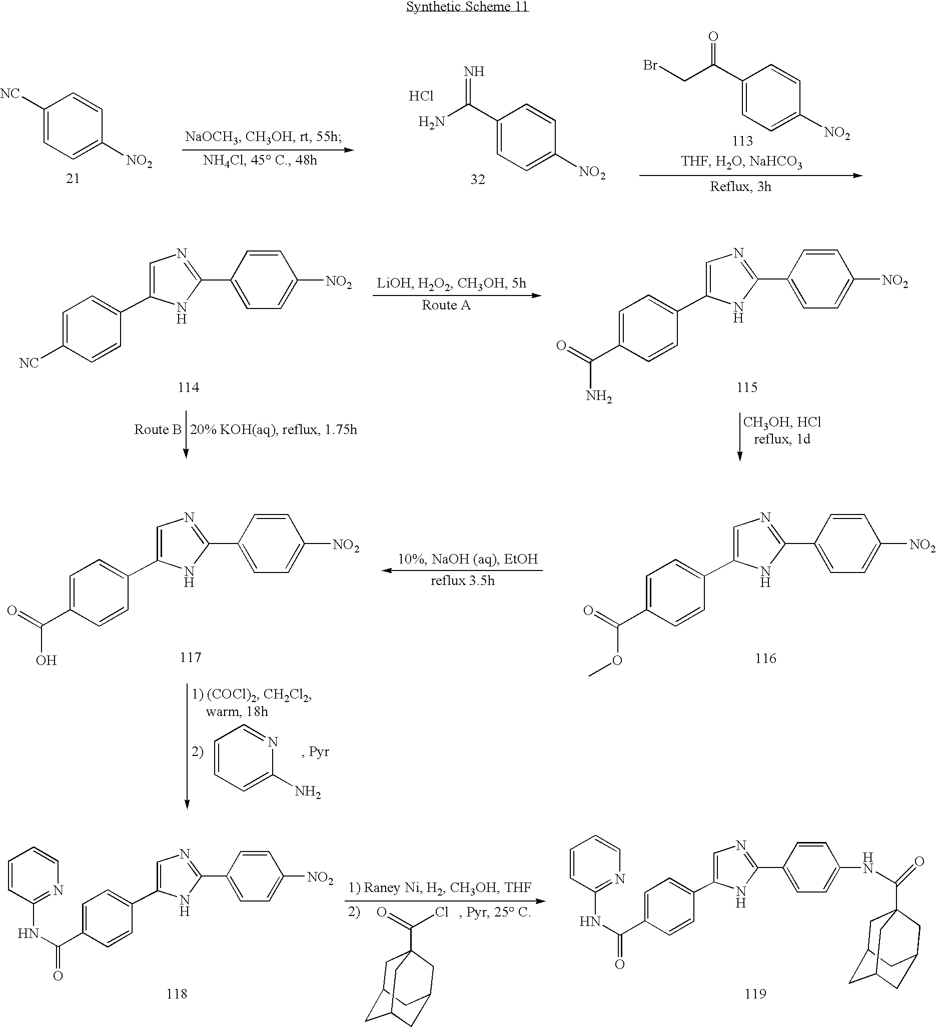

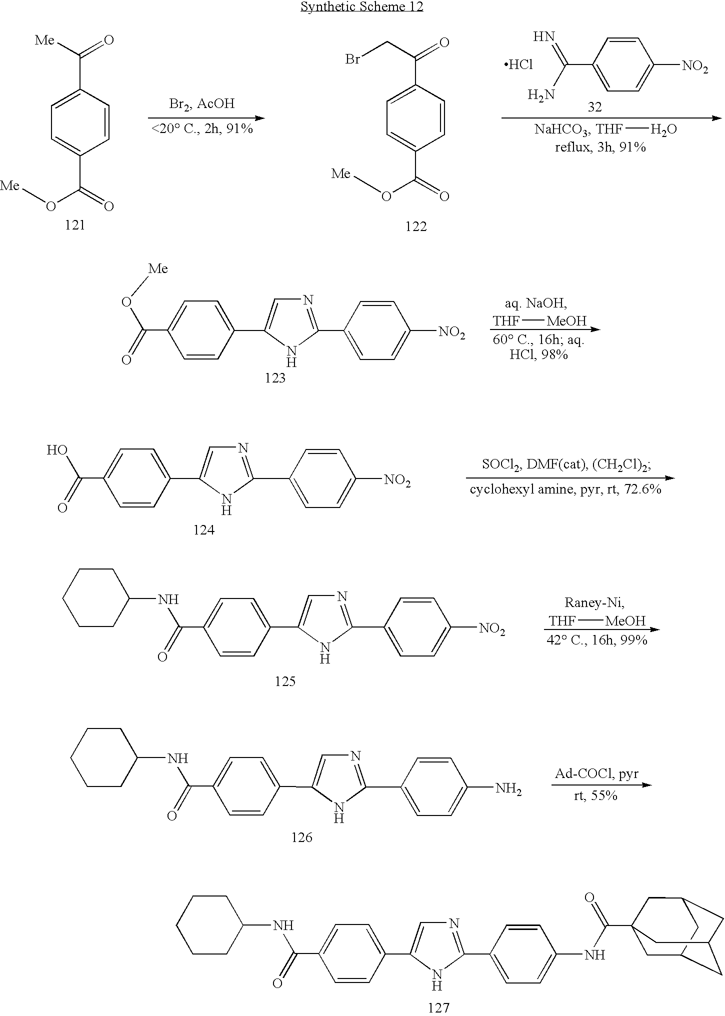

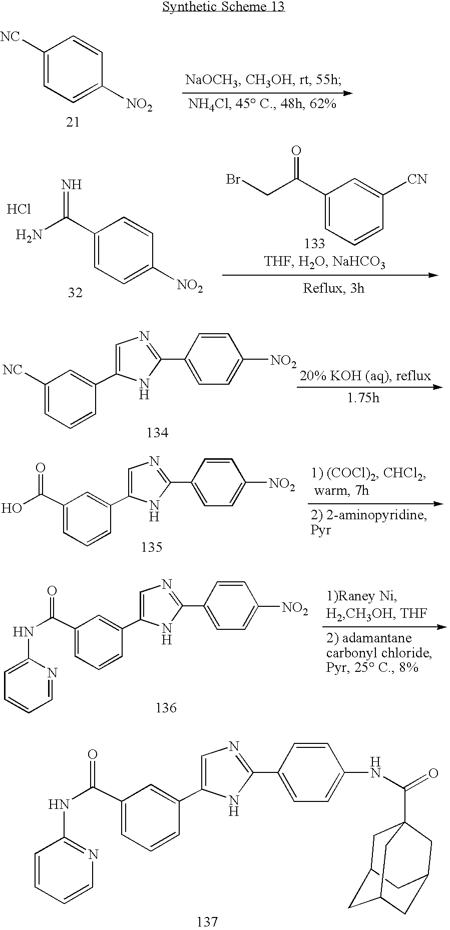

Accordingly, a preferred method of preparing a compound or salt thereof having the formula:

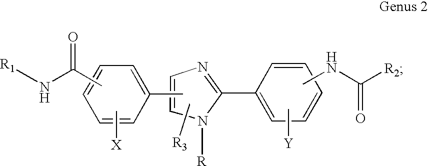

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl;

comprises the following steps: converting a Y-substituted-nitro-benzonitrile to a Y-substituted nitro-benzamidine; reacting the Y-substituted nitro-benzamidine with X-substituted cyano-phenacyl halide to form a species of the formula 92

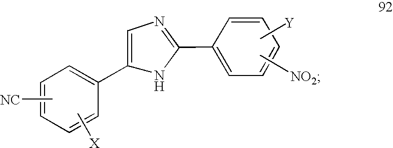

reducing the species of the formula 92 to form a species of the formula 93

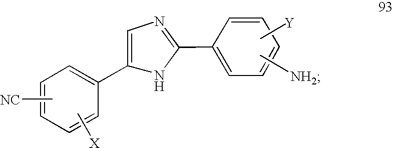

acylating the species of the formula 93 and subsequently performing a hydrolysis to form a species of the formula 94

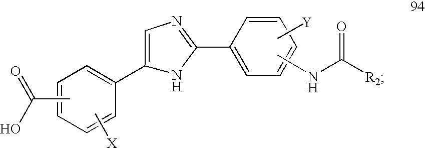

and aminating the species of the formula 94 to form a species of the formula 95

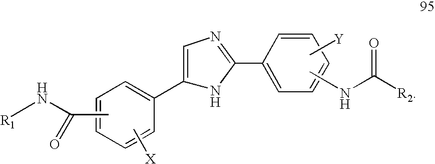

Accordingly, another preferred method of preparing a compound or salt thereof having the formula:

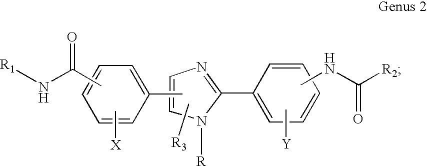

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl;

comprises the following steps: converting a Y-substituted nitro-benzonitrile to a Y-substituted nitro-benzamidine; converting a methyl X-substituted 4-acetyl benzoate to a methyl X-substituted 4-(alpha-bromoacetyl)benzoate; reacting the Y-substituted nitro-benzamidine with methyl X-substituted 4-(alpha-bromoacetyl)benzoate to form species of the formula 103

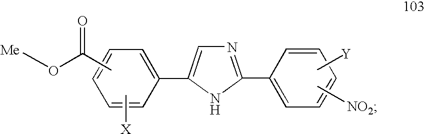

hydrolyzing the species of the formula 103 to form a species of the formula 104

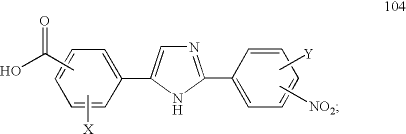

aminating the species of the following formula 104 to form a species of the formula 105

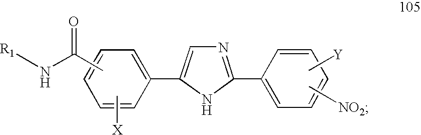

and reducing and amidating the formula 105 to form a species of the formula 106

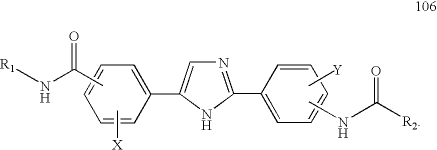

Synthesis of the Compounds of Genus 2

Synthetic Schemes 9-13 shows methods that can be used to prepare the compounds of Genus 2. One skilled in the art will appreciate that a number of different synthetic reaction schemes may be used to synthesize the compounds of Genus 2. Further, one skilled in the art will understand that a number of different solvents, coupling agents and reaction conditions can be used in the syntheses reactions to yield comparable results.

One skilled in the art will appreciate variations in the sequence and further, will recognize variations in the appropriate reaction conditions from the analogous reactions shown or otherwise known which may be appropriately used in the processes above to make the compounds of Synthetic Schemes 9-13.

In the processes described herein for the preparation of the compounds of Synthetic Schemes 9-13 of the preferred embodiments, the requirements for protective groups are generally well recognized by one skilled in the art of organic chemistry, and accordingly the use of appropriate protecting groups is necessarily implied by the processes of the schemes herein, although such groups may not be expressly illustrated. Introduction and removal of such suitable protecting groups are well known in the art of organic chemistry; see for example, T. W. Greene, “Protective Groups in Organic Synthesis”, Wiley (New York), 1981.

The products of the reactions described herein are isolated by conventional means such as extraction, distillation, chromatography, and the like.

Starting materials not described herein are available commercially, are known, or can be prepared by methods known in the art.

The salts of the compounds of Synthetic Schemes 9-13 described above are prepared by reacting the appropriate base or acid with a stoichiometric equivalent of the compounds of Synthetic Schemes 9-13.

Compounds of Genus 3

One family of small molecule IgE inhibitors is defined by the following genus (Genus 3):

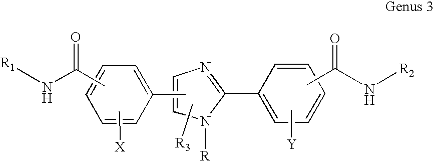

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

Compounds of Genus 3 may be synthesized by any conventional reactions known in the art. Examples of syntheses include the following reactions, designated Synthetic Scheme 14:

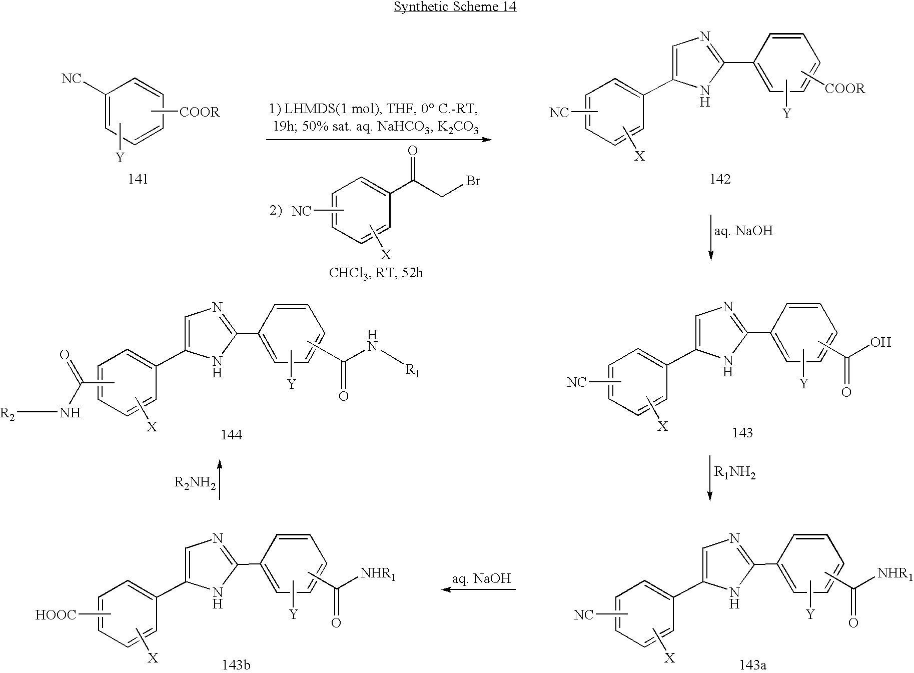

Accordingly, a preferred method of preparing a compound or salt thereof having the formula:

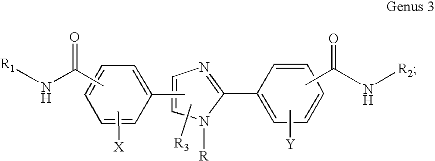

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl;

comprises the following steps: converting a Y-substituted-alkoxycarbonyl-benzonitrile to a Y-substituted alkoxycarbonyl-benzamidine; reacting the Y-substituted alkoxycarbonyl-benzamidine with X-substituted cyano-phenacyl halide to form a species of the formula 142

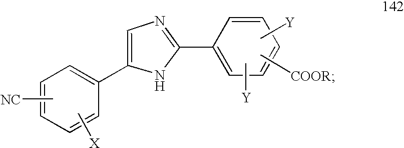

hydrolyzing the species of the formula 142 to form a species of the formula 143

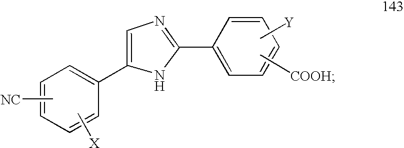

amidating the species of the formula 143 to form a species of the formula 143a

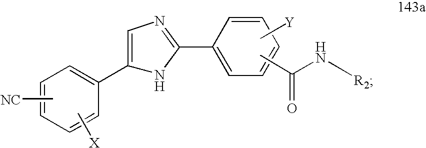

hydrolyzing the species of the formula 143a to form a species of the formula 43b

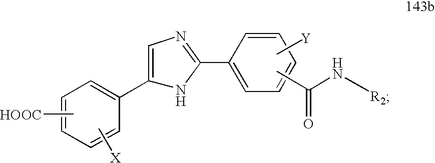

and amidating the species of the formula 143b to form a species of the formula 144

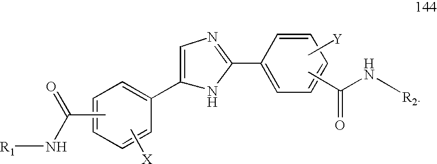

Synthesis of the Compounds of Genus 3

Synthetic Scheme 14 shows methods that can be used to prepare the compounds of Genus 3. One skilled in the art will appreciate that a number of different synthetic reaction schemes may be used to synthesize the compounds of Genus 3. Further, one skilled in the art will understand that a number of different solvents, coupling agents and reaction conditions can be used in the syntheses reactions to yield comparable results.

One skilled in the art will appreciate variations in the sequence and further, will recognize variations in the appropriate reaction conditions from the analogous reactions shown or otherwise known which may be appropriately used in the processes above to make the compounds of Synthetic Scheme 14.

In the processes described herein for the preparation of the compounds of Synthetic Scheme 14 of the preferred embodiments, the requirements for protective groups are generally well recognized by one skilled in the art of organic chemistry, and accordingly the use of appropriate protecting groups is necessarily implied by the processes of the schemes herein, although such groups may not be expressly illustrated. Introduction and removal of such suitable protecting groups are well known in the art of organic chemistry; see for example, T. W. Greene, “Protective Groups in Organic Synthesis”, Wiley (New York) 1981.

The products of the reactions described herein are isolated by conventional means such as extraction, distillation, chromatography, and the like.

Starting materials not described herein are available commercially, are known, or can be prepared by methods known in the art.

The salts of the compounds of Synthetic Scheme 14 described above are prepared by reacting the appropriate base or acid with a stoichiometric equivalent of the compounds of Synthetic Scheme 14.

Compounds of Genus 4

One family of small molecule IgE inhibitors is defined by the following genus (Genus 4):

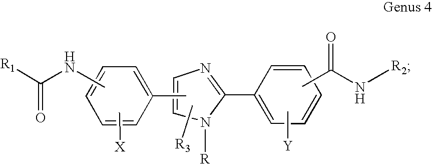

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alky, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl.

Compounds of Genus 4 may be synthesized by any conventional reactions known in the art. Examples of syntheses include the following reactions, designated Synthetic Scheme 15:

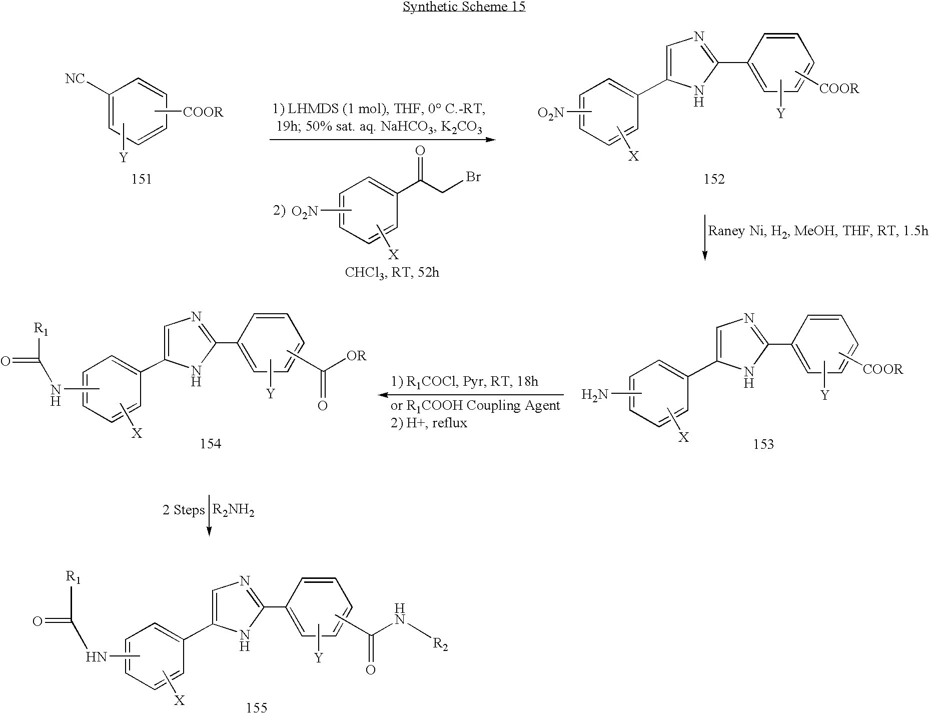

Accordingly, a preferred method of preparing a compound or salt thereof having the formula:

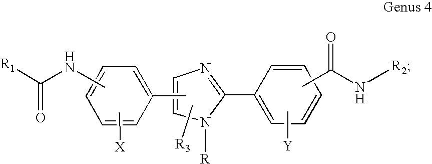

wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;

wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;

wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;

wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′ COR;, CN, CF₃, OCF₃, NO₂, NR′R′, NHCOR′ and CONR′R′;

wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and

wherein R″ is selected from the group consisting of C₁-C₉ alkyl, wherein said C₁-C₉ alkyl is selected from the group consisting of straight chain alkyl, branched alkyl, and cyclic alkyl;

comprises the following steps: converting a Y-substituted-alkoxycarbonyl-benzonitrile to a Y-substituted alkoxycarbonyl-benzamidine; reacting the Y-substituted alkoxycarbonyl-benzamidine with X-substituted nitro-phenacyl halide to form a species of the formula 152

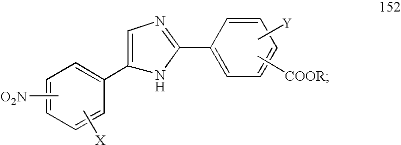

reducing the species of the formula 152 to form a species of the formula 153

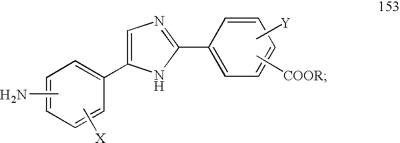

acylating the species of the formula 153 to form a species of the formula 154

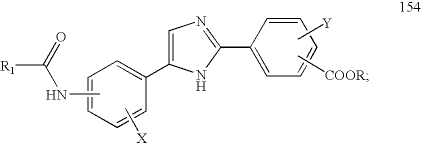

and amidating the species of the formula 154 to form a species of the formula 155

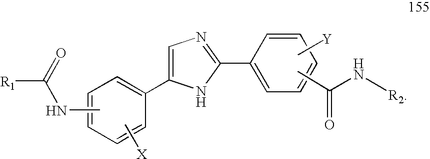

Synthesis of the Compounds of Genus 4

Synthetic Scheme 15 shows methods that can be used to prepare the compounds of Genus 4. One skilled in the art will appreciate that a number of different synthetic reaction schemes may be used to synthesize the compounds of Genus 4. Further, one skilled in the art will understand that a number of different solvents, coupling agents and reaction conditions can be used in the syntheses reactions to yield comparable results.

One skilled in the art will appreciate variations in the sequence and further, will recognize variations in the appropriate reaction conditions from the analogous reactions shown or otherwise known which may be appropriately used in the processes above to make the compounds of Synthetic Scheme 15.

In the processes described herein for the preparation of the compounds of Synthetic Scheme 15 of the preferred embodiments, the requirements for protective groups are generally well recognized by one skilled in the art of organic chemistry, and accordingly the use of appropriate protecting groups is necessarily implied by the processes of the schemes herein, although such groups may not be expressly illustrated. Introduction and removal of such suitable protecting groups are well known in the art of organic chemistry; see for example, T. W. Greene, “Protective Groups in Organic Synthesis”, Wiley (New York), 1981.

The products of the reactions described herein are isolated by conventional means such as extraction, distillation, chromatography, and the like.

Starting materials not described herein are available commercially, are known, or can be prepared by methods known in the art.

The salts of the compounds of Synthetic Scheme 15 described above are prepared by reacting the appropriate base or acid with a stoichiometric equivalent of the compounds of Synthetic Scheme 15.

In Genera 1-4, preferred substituents for R₁ and R₂ are independently selected from the following and similar substituents thereof:

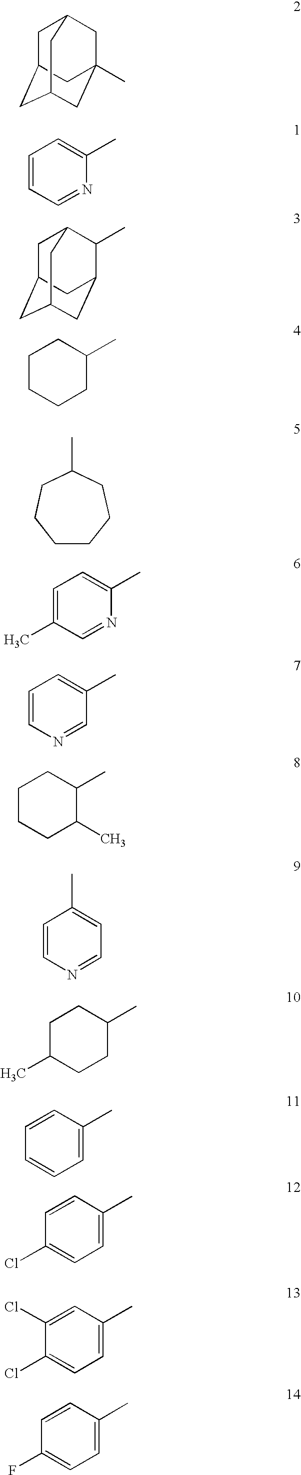

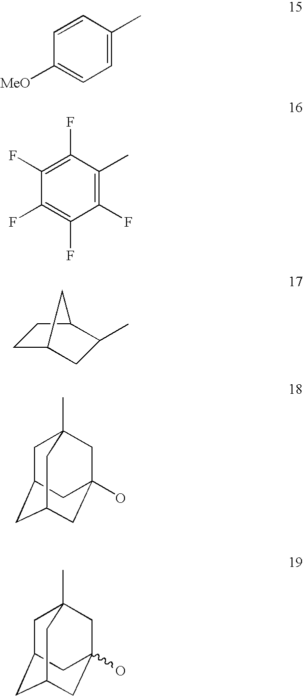

More preferably, substituents for R₁ and R₂ are selected from substituents 1-5 and 13.

### EXAMPLE 1

### Synthetic Scheme 2

2,5-Bis-(4-nitrophenyl)-1H-imidazole (22). To a solution of 4-nitrobenzonitrile (3.0 mmol, 444 mg) in dry THF (3 mL) was added lithium bistrimethylsilyl amide (1.0 M solution in THF, 3.6 mL) dropwise. The mixture was allowed to stir at room temperature for 18 hours and was then quenched with 50% saturated aqueous NaHCO₃ (6 mL). To this mixture was added K₂CO₃ (414 mg, 3 mmol) as a solid and CHCl₃ (10 mL) followed by 3-bromo-4′-nitroacetophenone (732 mg, 3 mmol) and the mixture stirred for 54 h at room temperature. The mixture was diluted with 40 mL CH₂Cl₂ and the organic layer separated and washed with aqueous saturated NaHCO₃ (30 mL) and aqueous saturated NaCl (30 mL) then dried over MgSO₄, filtered and concentrated. The resulting oily solid was purified by flash chromatography over silica using CH₂Cl₂/CH₃OH (19:1) as eluent to give the product as a yellow solid (150 mg, 0.5 mmol, 17%)

2,4-Bis-(4-aminophenyl)-1H-imidazole (23). To a solution of 2,4-di(4-nitrophenyl)-1H-imidazole (22) (150 mg, 0.48 mmol) in CH₃OH (15 mL) and THF (2.5 mL) was added Raney Ni and the system vacuum purged with H₂ three times. The mixture was stirred under H₂ gas at room temperature for 1.5 h and then filtered through celite. The filtrate was concentrated under reduced pressure to give a yellow residue (82 mg, 0.32 mmol, 67%) that was used without further purification.

N-{4-[5-(4-cyclohexylamino-phenyl)-1H-imidazol-2-yl]-phenyl}-cyclohexlamide (24). To a solution of 2,4-di(4-aminophenyl)-1H-imidazole (23) (82 mg, 0.32 mmol) in pyridine (5 mL) was added cyclohexane carboxylic acid chloride (2 eq, 86 ul, 94 mg, 0.64 mmol) and the mixture stirred at room temperature under inert atmosphere for 18 h. The mixture was poured into H₂O (125 mL) and stirred for 25 min. The resulting yellow precipitate was collected by filtration (97 mg) and a portion (40 mg) purified by flash chromatography over silica using CH₂Cl₂/CH₃OH (19:1) as eluent to give the product as a pale yellow solid (20 mg, 0.085 mmol, 27%). mp 335-337 C, ¹H-NMR (500 MHz, DMSO-d₆) d 12.37 (apparent d, 1H), 9.85 (apparent d, 2H), 7.88 (d, 2H, J=8.64 Hz), 7.75 (d 2H, J=8.66 Hz), 7.69 (d, 2H, J=8.45 Hz), 7.66 (apparent d), 7.60 (d, 2H, J=8.59), 2.34 (m, 2H), 1.78 (m, 8H), 1.66 (m, 3H), 1.42 (m, 4H), 1.26 (m, 6H); M/z=471.6 (M+); TLC silica Rf=0.43 19:1 dichloromethane/methanol; Anal. (C₂₉H₃₄N₄O₂) C, H, N

### Synthetic Scheme 3

4-Nitrobenzamidine HCl (32). (prepared by the known method Journal of Organic Chemistry 55, 7, 1990, 2003-2004) To a solution of 4-nitrobenzonitrile (10 g, 67.5 mmol) in dry methanol (90 ml) was added a solution of sodium methoxide (7.4 mmol, 400 mg) in dry methanol (7.4 mL) and the solution warmed until complete dissolution of the solid. The solution was stirred at room temperature for 55 h at which time solid NH₄Cl (3.69 g, 69 mmol) was added and the mixture heated at 45° C. for 48 h. The mixture was cooled to room temperature and the resulting solid collected by filtration, rinsed with acetone and dried to give the product as a yellow solid (3.7 g, 18.4 mmol). The crude product was used as is in subsequent steps.

2,5-bis(4-nitrophenyl)-1H-imidazole (34). (prepared by the known method Organic Process Research & Development 6, 2002, 682-683) To a solution of 4-nitrobenzamidine (32) (1 g, 5 mmol) in THF (8.5 mL) and H₂O (3 mL) was added NaHCO₃ (4×, 1.68 g, 20 mmol) and the solution was brought to a vigorous reflux. A solution of 4-nitrophenacyl bromide (33) (1.22 g, 5 mmol) in dry THF (2 mL) was added dropwise and the solution heated at reflux for 2 h. The mixture was cooled and the THF removed under reduced pressure to give a purple residue. The residue was dissolved in acetone (5 mL) and poured into H₂O (200 mL) and stirred for 20 min. The resulting solid was collected by filtration and dried to give the product as a purple solid. (1.05 g, 3.4 mmol, 68%) The crude product was used as is in subsequent steps.

4-(2-(4-aminophenyl)-1H-imidazol-5-yl)benzenamine (35). To a solution of 2,5-bis(4-nitrophenyl)-1H-imidazole (34) (1 g, 3.22 mmol) in CH₃OH (50 mL) was added an aqueous slurry of Raney Nickel. The mixture was vacuum purged 5 times with H₂ and stirred under an atmosphere of H₂ at room temperature for 3 h. The catalyst was removed by filtration through celite and the filtrate concentrated to give the product (0.851 g, 3.2 mmol, 100%). The product was used as is in subsequent steps.

N-{4-[5-(4-adamantylamino-phenyl)-1H-imidazol-2-yl]-phenyl}-adamantylamide (36). To a solution of 4-(2-(4-aminophenyl)-1H-imidazol-5-yl)benzenamine (35) (283 mg, 1.13 mmol) in dry pyridine (15 mL) was added adamantancarbonyl chloride (2.1 eq, 472 mg, 2.4 mmol) and the mixture stirred at room temperature for 18 h and then diluted with H₂O (55 mL). The resulting solid was collected by filtration, dried and purified by chromatography over silica (dichloromethane/methanol, 0-5% gradient, 30 min.) giving the product as a tan solid (95 mg, 0.17 mmol, 15%) Mp: 382° C. ¹H NMR (500 MHz, DMSO-d₆) δ12.44 (apparent d, 1H), 9.23(s, 1H), 9.10 (s, 1H), 7.91 (m, 2H), 7.65 (m, 4H), 2.03(bs, 4H), 1.92 (bs, 9H), 1.71(bs, 9H). EIMS m/z M⁺¹ 575.5. Anal. (C, H, N, +1 CH₃OH)

The following compounds were synthesized using the above procedure.

N-{4-[5-(4-cycloheptylamino-phenyl)-1H-imidazol-2-yl]-phenyl}-cycloheptylamide. Product as a brown solid (15 mg, 0.03 mmol, 2.7%) Mp: 318-320° C. ¹H NMR (500 MHz, DMSO-d₆) δ12.37 (apparent d, 1H), 9.90(s, 1H), 9.76 (s, 1H), 7.88 (d, J=9 Hz, 2H), 7.74 (d, J=9 Hz 2H), 7.66(m, 6H), 1.92 (bs, 9H), 1.86-1.45 (m, 26H). EIMS m/z M⁺¹ 499.6. Anal. (C, H, N, +2 H₂O)

N-{4-[2-(4-(4-fluorobenzoylamino)-phenyl)-3H-imidazol-4-yl]-phenyl}-4-fluoro-benzamide. Product as a green solid (18 mg, 0.04 mmol, 1.3%) Mp: 345° C. dec. ¹H NMR (400 MHz, DMSO-d₆) δ10.36 (apparent d, 2H), 8.06(m, 4H), 8.0 (m, 1H), 7.88 (m, 4H), 7.78 (m, 3H), 7.4 (m, 4H). EIMS m/z M⁺¹ 495.4. Anal. (C, H, N)

N-{4-[5-(4-cyclohexylamino-phenyl)-1H-imidazol-2-yl]-phenyl}-cyclohexylamide. Product as a yellow solid (95 mg, 0.04 mmol, 13%) Mp: 335-337° C. ¹H NMR (500 MHz, DMSO-d₆) δ12.43 (apparent d, 1H), 9.92(s, 1H), 9.9.78 (s, 1H), 7.91 (m, 1H), 7.71 (dd,J=5 Hz, 30 Hz, 4H), 7.66 (m, 4H), 2.36 (m, 1H), 1.78(m, 3H), 1.65(m, 1H), 1.42(m, 2H), 1.26(m, 3H). EIMS m/z M⁺¹ 471.3. Anal. (C, H, N)

N-{4-[2-(4-(2,4-dichlorobenzoylamino)-phenyl)-3H-imidazol-4-yl]-phenyl}-2,4-dichloro-benzamide. Product as a green solid (36 mg, 0.06 mmol, 1.9%) Mp: 310° C. ¹H NMR (400 MHz, DMSO-d₆) δ12.6 (1, 2H), 8.06(m, 4H), 10.54 (s, 1H), 10.42 (s, 1H), 8.42 (m, 2H), 7.9 (m, 4H), 7.86 (m, 5H), 7.82 (m, 2H), 7.73 (m, 1H). EIMS m/z M⁺¹ 595.9. Anal. (C, H, N)

N-{4-[5-(4-(2-methylcyclohexyl)-amino-phenyl)-1H-imidazol-2-yl]-phenyl}-(2-methylcyclohexyl)-amide. Product as a brown solid (32 mg, 0.06 mmol, 1.3%) Mp: 195-199° C. ¹H NMR (400 MHz, DMSO-d₆) δ12.46 (apparent d, 1H), 9.88(dd, 1H), 9.78 (d,J=70 Hz, 1H), 7.94 (dd,J=10 Hz, 70 Hz, 1H), 7.89 (dd,J=15 Hz, 70 Hz, 4H), 7.63 (m, 6H), 2.53 (m, 2H), 2.12(m, 2H), 1.71(m, 8H), 1.50(m, 6H), 1.30(m, 5H), 0.90(d ,J=10 Hz, 3H), 0.84(d ,J=5 Hz, 2H). EIMS m/z M⁺¹ 499.4. Anal. (C, H, N)

### Synthetic Scheme 4

Preparation of 2-(3-Nitrophenyl)-5-(4-nitrophenyl)-1H-imidazole (43): To a mixture of 3-nitrobenzamidine hydrochloride (42) (2.06 g, 10.2 mmol) and anhydrous NaHCO₃ (3.44 g, 41.0 mmol) THF (18 mL) and water (4.5 mL) were added and heated at reflux for 20 min. Then a solution of 4-nitrophenacyl bromide (33) (2.50 g, 10.2 mmol) in THF (4.5 mL) was added slowly over 6 min via syringe. After refluxing for an additional 3 h, the flask was removed from the oil-bath and cooled to about 30° C. and evaporated off THF in a rotary evaporator (with care). Water (50 mL) was added to the residue and stirred for 30 min. The brown gum was filtered, washed with water (3×25 mL) and dried in vacuum oven at 80° C. overnight. The light-brown gummy material (compound 43, 3.17 g, 99.7%) was used in the subsequent step without further purification.

Synthesis of 3-(5-(4-aminophenyl)-1H-imidazol-2-yl)benzenamine (44): The nitro compound (1.5 g, 4.8 mmol) was dissolved in MeOH-THF (4:1; 60 mL), and degassed (argon atmosphere). To that slurry of Raney-Nickel (in water) (1.0 mL) was added carefully. The system was flushed once with hydrogen gas from a balloon. The reaction was stirred at rt under hydrogen gas (balloon) for 15 h. The supernatant was passed through a pad of Celite. The reaction flask was rinsed with MeOH (25 mL) and the supernatant was passed through the Celite. The filtrates were concentrated in a rotary evaporator and dried in vacuum to obtain light-brown solids (1.20 g, 99%). The diamine 44 was used in the subsequent reaction.

Preparation of N-(4-(2-(3-(Picolinamido)phenyl)-1H-imidazol-5-yl)phenyl)picolinamide (45): To a solution of the diamine 44 (0.19 g, 0.76 mmol) in pyridine (4 mL) picoloyl chloride hydrochloride (0.43 g, 2.4 mmol) was added and stirred at rt overnight. The solvent was removed and the residue was stirred with sat'd NaHCO₃ (5 mL) to obtain slurry material. The solids were filtered, washed with water (5 mL) and dried to obtain crude diamide 45. The material was purified further by reverse-phase chromatography (Combiflash; solvent mixture: CH₃CN/H₂O). The pure fractions were combined and evaporated off the volatiles (mostly the CH₃CN). Then sat'd NaHCO₃ (10 mL) was added and solids started to precipitate. The solids were filtered, washed with water (2×10 mL) and dried in vacuum oven at 80° C. overnight to obtain pure diamide 45 (0.052 g, 14.9%); mp 205-8° C. ¹H NMR (DMSO-d₆, δ in ppm): 10.76 (s, 1 H), 10.69 (s, 1 H), 8.71 (d, J=4.8 Hz, 1 H), 8.69 (d, J=4.8 Hz, 1 H), 8.61 (s, 1 H), 8.15-8.11 (m, 2 H), 8.05 (dd, J=7.6, 1.6 Hz, 1 H), 8.01 (dd, J=7.6, 1.6 Hz, 1 H), 7.93 (d, J=8.8, Hz, 2 H), 7.84 (s, 1 H), 7.81 (d, J=8.8 Hz, 2 H), 7.72 (d, J=8.8 Hz, 1 H), 7.70 (d, J=7.6 Hz, 1 H), 7.67-7.61 (m, 2 H), 7.45 (t, J=8.0 Hz, 1 H). MS: [EI] m/e 461.4 [M+H]⁺. Anal: (C₂₇H₂₀N₆O₂-0.74 H₂O-0.74 CF₃CO₂H) C, H, N.

### Synthetic Scheme 5

Preparation of 2,5-bis(3-nitrophenyl)-1H-imidazole (53): To a mixture of 3-nitrobenzamidine hydrochloride (42) (2.06 g, 10.2 mmol) and anhydrous NaHCO₃ (3.44 g, 41.0 mmol) THF (18 mL) and water (4.5 mL) were added, and heated at reflux for 20 min. Then a solution of 3-nitrophenacyl bromide (51) (2.50 g, 10.2 mmol) in THF (4.5 mL) was added slowly over 6 min via syringe. After refluxing for an additional 3 h, the flask was removed from the oil-bath and cooled to about 30° C. and evaporated off THF in a rotary evaporator (with care). Water (50 mL) was added to the residue and stirred for 30 min. The brown precipitates were filtered, washed with water (3×25 mL) and dried in vacuum oven at 80° C. overnight. The light-brown solids (compound 53, 3.00 g, 94.4%) were used in the subsequent step without further purification.

Synthesis of 3-(5-(3-aminophenyl)-1H-imidazol-2-yl)benzenamine (54): The nitro compound (1.5 g, 4.8 mmol) was dissolved in MeOH-THF (4:1; 60 mL), and degassed (argon atmosphere). To that slurry of Raney-Nickel (in water) (1.0 mL) was added carefully. The system was flushed once with hydrogen gas from a balloon. The reaction was stirred at rt under hydrogen gas (balloon) for 15 h. The supernatant was passed through a pad of Celite. The reaction flask was rinsed with MeOH (25 mL) and the supernatant was passed through the Celite. The filtrates were concentrated in a rotary evaporator and dried in vacuum to obtain light-brown solids (1.12 g, 92.5%). The diamine 54 was used in the next reaction.

Preparation of N-(3-(5-(3-(picolinamido)phenyl)-1H-imidazol-2-yl)phenyl)picolinamide (55): To a solution of the diamine 54 (0.19 g, 0.76 mmol) in pyridine (4 mL) picoloyl chloride hydrochloride (0.43 g, 2.4 mmol) was added and stirred at rt overnight. The solvent was removed and the residue was stirred with sat'd NaHCO₃ (5 mL) to obtain slurry material. The solids were filtered, washed with water (5 mL), and dried to obtain crude diamide 55. The material was purified further by reverse-phase chromatography (Combiflash; solvent mixture: CH₃CN/H₂O). The pure fractions were combined and evaporated off the volatiles (mostly the CH₃CN). Then sat'd NaHCO₃ (10 mL) was added and solids started to precipitate. The solids were filtered, washed with water (2×10 mL) and dried in vacuum oven at 80° C. overnight to obtain pure diamide 55 (0.061 g, 17.4%); mp 208-10° C. ¹H NMR (DMSO-d₆, δ in ppm): 10.73 (s, 1 H), 10.60 (s, 1 H), 8.77 (d, J=4.0 Hz, 2 H), 8.59 (s, 1 H), 8.38 (s, 1 H), 8.21 (apparent dd, J=8.0, 2.8 Hz, 2 H), 8.10 (apparent dt, J=7.6, 1.6 Hz, 2 H), 7.86 (d, J=8.0, Hz, 1 H), 7.79-7.69 (m, 4 H), 7.76 (s, 1 H), 7.64 (d, J=7.6 Hz, 1 H), 7.47 (d, J=8.0 Hz, 1 H), 7.38 (d, J=8.0 Hz, 1 H). MS: [EI] m/e 461.4 [M+H]⁺. Anal: (C₂₇H₂₀N₆O₂-1.29 H₂O-0.04 CF₃CO₂H) C, H, N.

The following compound was prepared using above route.

N-(3-(5-(3-(1-Adamantanamido)phenyl)-1H-imidazol-2-yl)phenyl)-1-adamantanecarboxamide: mp 261-3° C. A mixture of two sets of amide and some aromatic proton chemical shifts were seen. ¹H NMR (DMSO-d₆, δ in ppm): 9.43 (s, 0.3 H), 9.35 (s, 0.4 H), 9.33 (s, 0.7 H), 9.24 (s, 0.6 H), 8.22 (br. s, 1 H), 8.15 (br. s, 1 H), 7.97-7.94 (m, 2 H), 7.68-7.58 (m, 3 H), 7.51-7.49 (m, 1 H), 7.43 (d, J=8.0 Hz, 1 H), 2.03 (br. s, 6 H), 1.94 (br. s, 6 H), 1.92 (br. s, 6 H), 1.72 (br. s, 12 H). MS: [EI] m/e 575.8 [M+H]⁺. Anal: (C₃₇H₄₂N₄O₂-0.31 H₂O-0.43 CH₃OH-0.33 CF₃CO₂H) C, H, N.

### Synthetic Scheme 6

Preparation of 5-(3-nitrophenyl)-2-(4-nitrophenyl)-1H-imidazole (63): To a mixture of 4-nitrobenzamidine hydrochloride (32) (2.06 g, 10.2 mmol) and anhydrous NaHCO₃ (3.44 g, 41.0 mmol) THF (18 mL) and water (4.5 mL) were added and heated at reflux for 20 min. Then a solution of 3-nitrophenacyl bromide (51) (2.50 g, 10.2 mmol) in THF (4.5 mL) was added slowly over 6 min via syringe. After refluxing for an additional 3 h, the flask was removed from the oil-bath and cooled to about 30° C. and evaporated off THF in a rotary evaporator (with care). Water (50 mL) was added to the residue and stirred for 30 min. The brown precipitates were filtered, washed with water (3×25 mL) and dried in vacuum oven at 80° C. overnight. The medium-brown solids (compound 63, 3.16 g, 99.4%) were used in the subsequent step without further purification.

Synthesis of 3-(2-(4-aminophenyl)-1H-imidazol-5-yl)benzenamine (64): The nitro compound (1.5 g, 4.8 mmol) was dissolved in MeOH-THF (4:1; 60 mL), and degassed (argon atmosphere). To that slurry of Raney-Nickel (in water) (1.0 mL) was added carefully. The system was flushed once with hydrogen gas from a balloon. The reaction was stirred at rt under hydrogen gas (balloon) for 15 h. The supernatant was passed through a pad of Celite. The reaction flask was rinsed with MeOH (25 mL) and the supernatant was passed through the Celite. The filtrates were concentrated in a rotary evaporator and dried in vacuum to obtain light-brown solids (1.20 g, 99%). The diamine 64 was used in the next reaction.

Preparation of N-(3-(2-(4-(picolinamido)phenyl)-1H-imidazol-5-yl)phenyl)picolinamide (65): To a solution of the diamine 64 (0.19 g, 0.76 mmol) in pyridine (4 mL) picoloyl chloride hydrochloride (0.43 g, 2.4 mmol) was added and stirred at rt overnight. The solvent was removed and the residue was stirred with sat'd NaHCO₃ (5 mL) to obtain slurry material. The solids were filtered, washed with water (5 mL) and dried to obtain the crude dianide 45. The material was purified further by reverse-phase chromatography (Combiflash; solvent mixture: CH₃CN/H₂O). The pure fractions were combined and evaporated off the volatiles (mostly the CH₃CN). Then sat'd NaHCO₃ (10 mL) was added and solids started to precipitate. The solids were filtered, washed with water (2×10 mL) and dried in vacuum oven at 80° C. overnight to obtain pure diamide 65 (0.185 g, 52.9%); mp 255-7° C. ¹H NMR (DMSO-d₆, δ in ppm): 10.76 (s, 1 H), 10.59 (s, 1 H), 8.76 (d, J=4.8 Hz, 2 H), 8.35 (br. s, 1 H), 8.20 (dd, J=7.6, 3.6 Hz, 1 H), 8.18 (dd, J=7.2, 4.0 Hz, 1 H), 8.09 (t, J=7.6 Hz, 2 H), 8.04 (br. s, 4 H), 7.78 (d, J=7.6 Hz, 1 H), 7.70 (s, 1 H), 7.69 (apparent t, J=6.0 Hz, 2 H), 7.62 (d, J=7.2, Hz, 1 H), 7.38 (t, J=8.0 Hz, 1 H). MS: [EI] m/e 461.4 [M+H]⁺. Anal: (C₂₇H₂₀N₆O₂-0.41 H₂O-0.21 CF₃CO₂H) C, H, N.

The following compound was synthesized using above route.

N-(4-(5-(3-(1-Adamantanamido)phenyl)-1H-imidazol-2-yl)phenyl)-1-adamantanecarboxamide: mp 247-9° C. ¹H NMR (DMSO-d₆, δ in ppm): 9.26 (s, 1 H), 9.19 (s, 1 H), 8.08 (s, 1 H), 7.93 (d, J=8.4 Hz, 2 H), 7.79 (d, J=8.0, Hz, 2 H), 7.65 (br. s, 1 H), 7.59 (dd, J=8.0, 1.0 Hz, 1 H), 7.48 (d, J=7.6 Hz, 1 H), 7.28 (d, J=8.0 Hz, 1 H), 2.03 (br. s, 6 H), 1.93 (br. s, 12 H), 1.72 (br. s, 12 H). MS: [EI] m/e 575.8 [M+H]⁺. Anal: (C₃₇H₄₂N₄O₂-0.18 H₂O-0.24 CH₃OH-0.30 CF₃CO₂H) C, H, N.

### Synthetic Scheme 8

4-Nitrobenzamidine HCl (42). (prepared by the known method Journal of Organic Chemistry 55, 7, 1990, 2005-2004) To a solution of 4-nitrobenzonitrile (25.5 g, 172 mmol) in dry methanol (230 ml) was added a solution of sodium methoxide (1 g, 18.5 mmol) and the solution warmed until complete dissolution of the solid. The solution was stirred at room temperature for 55 h at which time solid NH₄Cl (9.5 g, 177 mmol) was added and the mixture heated at 45° C. for 48 h. The mixture was cooled to room temperature and the resulting solid collected by filtration, rinsed with acetone and dried to give the product as a yellow solid (21.6 g, 107 mmol, 62%). The crude product was used as is in subsequent steps.

4-[2-(4-Nitro-phenyl)-3H-imidazol-4-yl]-phenylamine (85). (prepared by the known method Organic Process Research & Development 6, 2002, 682-683) To a solution of 4-nitrobenzamidine (42) (3.18 g, 14 mmol) in THF (48 mL) and H₂O (14 mL) was added NaHCO₃ (4×, 9.4 g, 56 mmol) and the solution was brought to a vigorous reflux. A solution of 4-(2-chloroacetyl)-acetanilide (83) (3 g, 14 mmol) in dry THF (25 mL) was added dropwise and the solution heated at reflux for 4 h. The mixture was cooled and the THF removed under reduced pressure to give a brown residue (84).

The residue was suspended in 5M HCl (aq, 150 mL) and the resulting mixture heated at reflux for 1 h. During this time the solid turned bright yellow. The mixture was carfully neutralized with NaHCO₃ and the brown solid collected by filtration and dried under vacuum. The crude product was used as is in subsequent steps.

Cyclohexanecarboxylic acid-{4-[2-(4-nitro-phenyl)-3H-imidazol-4-yl]-phenyl}-amide (86). To a solution of 4-[2-(4-Nitro-phenyl)-3H-imidazol-4-yl]-phenylamine (85) (4 g, 14.3 mmol) in dry pyridine (200 mL) was added cyclohexancarboxylic acid chloride (1.1 eq, 2.2 g, 2.02 ml, 15 mmol) and the mixture stirred at room temperature for 3 h. The pyridine was removed under reduced pressure and the black residue diluted with saturated NaHCO₃. The resulting black tar/oil was collected by filtration and allowed to dry giving a solid that was broken up by sonication in water. (4.67 g, 13 mmol, 91%). The crude product was used as is in subsequent steps.

2-Methyl-cyclohexanecarboxylic acid {4-[5-(4-cyclohexylamino-phenyl)-1H-imidazol-2-yl]-phenyl}-amide (88). To a solution of Cyclohexanecarboxylic acid-{4-[2-(4-nitro-phenyl)-3H-imidazol-4-yl]-phenyl}-amide (86) (0.36 g, 1.0 mmol) in methanol/THF (10 mL; 1 mL) was added Raney nickel and the solution vacuum purged 5× with H₂ gas. The mixture was stirred under H₂ for 3.5 h and filtered through celite and concentrated under reduced pressure to give a brown foam solid residue that was used as is in the coupling step.

The residue was dissolved in dry pyridine (5mL) and 2-methylcyclohexanecarboxylic acid chloride (1.0 mmol, 0.162 g) and the solution stirred for 15 h. The pyridine was removed under reduced pressure and the resulting black residue sonicated in saturated NaHCO₃ (10 mL) followed by H₂O to give a solid that was collected by filtration and dried in vacuo. The crude solid was purified by flash chromatography using dichloromethane/methanol (0-5% gradient) over silica to give the product as a white solid. (41.5 mg, 0.087 mmol, 9%). Mp: 310° C. ¹H NMR (400 MHz, DMSO-d₆) δ12.48 (bs, 1H), 9.94(s, 0.3H), 9.87 (s, 1H), 9.80 (s, 1H), 7.76 (d, J=10.5 Hz, 2H), 7.71(m, 5H), 17.61 (d, 10.5 Hz, 2H), 7.57 (bs, 1H), 2.44 (m, 1H), 2.33 (m, 2H), 1.73 (m, 10H), 1.4 (m, 13H), 0.94 (d, J=10 Hz, 3H), 0.89 (d, J=10 Hz, 1H). EIMS m/z M⁺¹ 485.4. Anal. (C, H, N, +2 H₂O)

The following compounds were synthesized in a manner similar to that described above.

N-{4-[5-(4-(2-methylcyclohexyl)-amino-phenyl)-1H-imidazol-2-yl]-phenyl}-(4-methylcyclohexyl)-amide. Reverse phase chromatography over C18 using H₂O/ACN/TFA as eluent to yield the product as a tan solid (132 mg, 0.26 mmol, 32%) Mp: 205-209° C. ¹H NMR (400 MHz, DMSO-d₆) δ12.43 (bs, 1H), 9.85(m, 2H), 7.89 (d, J=4 Hz, 2H), 7.69 (m, 8H), 2.56 (m, 1H), 2.42 (m, 1H), 2.1 (m, 1H), 1.70(m, 8H), 1.55(m, 12H), 0.94(m, 8H). EIMS m/z M⁺¹ 499.6. Anal. (C, H, N)

N-(4-(5-(4-adamantylamidophenyl)-1H-imidazol-2-yl)phenyl)picolinamide. Product as a green solid (33 mg, 0.064 mmol, 8%) Mp: 341° C. ¹H NMR (400 MHz, DMSO-d₆) δ10.75 (s, 1H), 9.15(apparent d, 1H), 8.76 (m, 1H), 8.18 (m, 1H), 8.09 (m, 1H), 8.00 (m, 4H), 7.77 (m, 1H), 7.69(m, 4H), 2.03(bs, 3H), 1.92 (m, 6H), 1.72(bs, 6H). EIMS m/z M⁺¹ 518.4. Anal. (C, H, N)

N-(4-(5-(4-adamantylamidophenyl)-1H-imidazol-2-yl)phenyl)-4-methylcyclohexanecarboxamide. Reverse phase chromatography over C18 using H₂O/ACN/TFA as eluent to yield the product as a green solid (89 mg, 0.26 mmol, 22%) Mp: 215-217° C. ¹H NMR (400 MHz, DMSO-d₆) δ14.13 (bs, 1H), 10.18 (apparent d, J=36 Hz, 1H), 9.29 (s, 1H), 8.02 (m, 3H), 7.83 (m, 6H), 2.58 (m, 1H), 2.14 (m, 1H), 2.04 (s, 3H), 1.92(s, 6H), 1.68 (m, 10H), 1.53 (m, 3H), 1.30 (m, 3H), 0.87 (m, 3H). EIMS m/z M⁺¹ 537.6. Anal. (C, H, N +1TFA)

N-(4-(5-(4-adamantylamidophenyl)-1H-imidazol-2-yl)phenyl)-2-methylcyclohexanecarboxamide. Reverse phase chromatography over C18 using H₂O/ACN/TFA as eluent to yield the product as a green solid (14mg, 0.026 mmol, 3%) Mp: 231-232° C. ¹H NMR (400 MHz, DMSO-d₆) δ12.50 (bs, 1H), 9.91 (apparent d, J=42 Hz, 1H), 9.11 (s, 1H), 7.9 (d, J=8 Hz, 2H), 7.74 (d, J=8 Hz, 2H), 7.68 (m, 3H), 7.59 (bs, 1H), 2.54 (m, 1H), 2.12(bs, 1H), 1.91 (d,J=4 Hz, 6H), 1.69 (m, 9H), 1.50 (m, 3H), 1.30 (m, 2H), 0.87 (m, 3H). EIMS m/z M⁺¹ 537.6. Anal. (C, H, N)

N-(4-(5-(4-adamantylamidophenyl)-1H-imidazol-2-methylcyclohexanecarboxamide. Reverse phase chromatography over C18 using H₂O/ACN/TFA as eluent to yield the product as a green solid (68 mg, 0.13 mmol, 17%) Mp: 222-225° C. ¹ H NMR (400 MHz, DMSO-d₆) δ14.32 (bs, 1H), 10.16 (s, 1H), 9.29 (s, 1H), 8.01 (s, 3H), 7.81 (d, 6H), 2.53 (m, 1H), 2.04 (bs, 3H), 1.90 (m, 8H), 1.66(m, 18H). EIMS m/z M⁺¹ 537.6. Anal. (C, H, N +1 TFA)

N-(4-(2-(4-adamantylamidophenyl)-1H-imidazol-5-yl)phenyl)cyclohexanecarboxamide. Reverse phase chromatography over C18 using H₂O/ACN/TFA as eluent to yield the product as a white solid (7 mg, 0.013 mmol, 1%) Mp: 240-241° C. ¹H NMR (400 MHz, DMSO-d₆) δ12.46 (bs, 1H), 9.80 (s, 1H), 9.23 (s, 1H), 7.91 (d, J=12 Hz, 2H), 7.76 (m, 4H), 7.61 (d, J=8 Hz, 3H), 2.31 (m, 1H), 2.03 (bs, 3H), 1.92(bs, 7H), 1.73 (m, 12H), 1.42 (m, 2H), 1.23 (m, 4H). EIMS m/z M⁺¹ 523.6. Anal. (C, H, N)

N-(4-(5-(4-(cyclohexanecarboxamido)phenyl)-1H-imidazol-2-yl)phenyl)picolinamide. Reverse phase chromatography over C18 using H₂O/ACN/TFA as eluent to yield the product as a white solid (115 mg, 0.247 mmol, 25%) Mp: 264-265° C. ¹H NMR (400 MHz, DMSO-d₆) δ12.04 (bs, 1H), 10.59 (s, 1H), 9.79 (d, J=1 Hz, 21H), 9.13 (d, J=1.6 Hz, 1H), 8.77 (dd, J=4.8 Hz, 1.6 Hz, 1H), 8.32 (dt, J=8 Hz, 4 Hz, 2 Hz, 1H), 7.99 (d, J=4 Hz, 2H), 7.88 (d, J=8 Hz, 2H), 7.66 (m, 11H), 1.73 (m, 12H), 6.61 (d, J=8 Hz, 2H), 5.31 (s, 2H), 2.32 (m, 2H), 1.78 (m, 7H), 1.65 (m, 2H), 1.43 (q, J=8 Hz, 20 Hz, 4H), 1.26 (m, 6H) EIMS m/z M⁺¹ 466.6. Anal. (C, H, N)

N-(4-(5-(4-(cyclohexanecarboxamido)phenyl)-1H-imidazol-2-yl)phenyl)-2-methylcyclohexylamide. Reverse phase chromatography over C18 using H₂O/ACN/TFA as eluent to yield the product as a tan solid (45 mg, 0.093 mmol, 9%) Mp: 190-193° C. ¹H NMR (400 MHz, DMSO-d₆) δ10.10 (s, 1H), 9.98 (s, 1H), 7.97 (m, 3H), 7.80 (m, 4H), 7.72 (m, 2H), 2.58 (m, 1H), 2.35 (m, 1H), 2.14 (m, 1H), 1.75 (m, 9H), 1.43 (m, 12H), 6.61 (d, J=8 Hz, 2H), 5.31 (s, 2H), 2.32 (m, 2H), 1.78 (m, 7H), 1.65 (m, 2H), 1.43 (m, 11H), 0.87 (m, 3H). EIMS m/z M⁺¹ 485.6. Anal. (C, H, N)

N-(4-(5-(4-(cyclohexanecarboxamido)phenyl)-1H-imidazol-2-yl)phenyl)cycloheptylamide. Reverse phase chromatography over C18 using H₂O/ACN/TFA as eluent to yield the product as a tan solid (44 mg, 0.091 mmol, 9%) Mp: 325° C. ¹H NMR (400 MHz, DMSO-d₆) δ9.98 (s, 1H), 9.85 (s, 1H), 7.92 (d, J=8 Hz, 3H), 7.72 (m, 9H), 2.33 (m, 1H), 1.9-1.1(m, 30H). EIMS m/z M+1 485.4. Anal. (C, H, N)

4-chloro-N-(4-(5-(4-(cyclohexanecarboxamido)phenyl)-1H-imidazol-2-yl)phenyl)benzamide. Reverse phase chromatography over C18 using H₂O/ACN/TFA as eluent to yield the product as a tan solid (15 mg, 0.030 mmol, 1%) Mp: 342° C. ¹H NMR (400 MHz, DMSO-d₆) δ12.51 (s, 1H), 12.37 (s, 0.3H), 10.43 (s, 1H), 9.87 (s, 0.3H) 9.78 (s, 0.7H), 8.00 (m, 5H), 7.87(d, J=8.8 Hz, 2H), 7.76 (d, J=8.8 Hz, 2H), 7.65 (m, 5H), 2.32 (m, 1H), 1.79 (m, 4H), 1.66 (m, 1H), 1.42 (m, 2H), 1.25 (m, 3H). EIMS m/z M⁺¹ 499.4. Anal. (C, H, N)

3,4-chloro-N-(4-(5-(4-(cyclohexanecarboxamido)phenyl)-1H-imidazol-2-yl)phenyl)benzamide. Reverse phase chromatography over C18 using H₂O/ACN/TFA as eluent to yield the product as a tan solid (81 mg, 0.15 mmol, 15%) Mp: 275° C. ¹H NMR (400 MHz, DMSO-d₆) δ9.80 (s, 1H), 8.25 (d, J=4 Hz, 1H), 7.97 (m, 3H), 7.85 (m, 3H), 7.75 (d, J=8.8 Hz, 2H), 7.62 (d, J=8.8 Hz, 3H), 2.32 (m, 1H), 1.79 (m, 5H), 1.66 (m, 1H), 1.42 (m, 2H), 1.26 (m, 4H). EIMS m/z M⁺¹ 533.4. Anal. (C, H, N)

N-(4-(5-(4-(4-methylcyclohexanecarboxamido)phenyl)-1H-imidazol-2-yl)phenyl)cycloheptanecarboxamide. Reverse phase chromatography over C18 using H₂O/ACN/TFA as eluent to yield the product as a tan solid (132 mg, 0.265 mmol, 33%) Mp: 292-293° C. ¹ H NMR (400 MHz, DMSO-d₆) δ9.79 (m, 2H), 7.94 (d, J=7.6 Hz, 0.5H), 7.89 (d, J=8.4 Hz, 2H), 7.75 (d, J=8.8 Hz, 2H), 7.68 (d, J=8.8 Hz, 3H), 7.60(d, J=8.8 Hz, 2H), 7.34(s, 0.2H), 2.43 (m, 1H), 1.9-1.4 (m, 22H), 0.90 (m, 4H). EIMS m/z M⁺¹ 499.4. Anal. (C, H, N)

N-(4-(2-(4-adamantylamidophenyl)-1H-imidazol-5-yl)phenyl)-4-methylcyclohexanecarboxamide. Reverse phase chromatography over C18 using H₂O/ACN/TFA as eluent to yield the product as a brown solid (97 mg, 0.181 mmol, 23%) Mp: 237-240° C. ¹H NMR (400 MHz, DMSO-d₆) δ12.43 (s, 0.7H), 12.30 (s, 0.3H), 9.79 (m, 2H), 7.94 (d, J=8.8 Hz, 0.5H), 7.89 (d, J=8.8 Hz, 2H), 7.75 (d, J=8.8 Hz, 3H), 7.68 (d, J=8.4 Hz, 3H), 7.60 (d, J=8.8 Hz, 2H), 2.43 (m, 1H), 1.69 (m, 22H), 0.92 (m, 4H). EIMS m/z M⁺¹ 537.6. Anal. (C, H, N)

N-(4-(5-(4-(4-methylcyclohexanecarboxamido)phenyl)-1H-imidazol-2-yl)phenyl)picolinamide. Reverse phase chromatography over C18 using H₂O/ACN/TFA as eluent to yield the product as a brown solid (122 mg, 0.254 mmol, 31%) Mp: 181-183° C. ¹H NMR (400 MHz, DMSO-d₆) δ10.98 (s, 1H), 10.01 (s, 0.3H), 9.95 (s, 0.7H), 8.78 (dt, J=1.2 Hz, 4.4 Hz, 1H), 8.18 (m, 3H), 8.09 (m, 4H), 7.82 (d, J=8.8 Hz, 2H), 7.72 (m, 3H), 2.46 (m, 1H), 1.77 (m, 4H), 1.50 (m, 6H), 0.92 (m, 4H). EIMS m/z M⁺¹ 480.4. Anal. (C, H, N)

N-(4-(5-(4-(4-methylcyclohexanecarboxamido)phenyl)-1H-imidazol-2-yl)phenyl)benzamide. Reverse phase chromatography over C18 using H₂O/ACN/TFA as eluent to yield the product as a white solid (135 mg, 0.282 mmol, 36%) Mp: 287-290° C. ¹H NMR (400 MHz, DMSO-d₆) δ10.37 (s, 1H), 9.81 (s, 0.3H), 9.75 (s, 0.7H), 7.97 (m, 5H), 7.88 (d, J=8.8 Hz, 3H), 7.75 (m, 3H), 7.57 (m, 8H), 2.44 (m, 1H), 1.79 (m, 4H), 1.52 (m, 7H), 0.92 (m, 5H). EIMS m/z M⁺¹ 479.4. Anal. (C, H, N)

N-(4-(5-(4-(4-methylcyclohexanecarboxamido)phenyl)-1H-imidazol-2-yl)phenyl)-4-fluorobenzamide. Reverse phase chromatography over C18 using H₂O/ACN/TFA as eluent to yield the product as a tan solid (102 mg, 0.205 mmol, 26%) Mp: 303-305° C. ¹H NMR (400 MHz, DMSO-d₆) δ10.38 (bs, 1H), 9.80 (m, 1H), 8.05 (m, 3H), 7.98 (d, J=8.4 Hz, 3H), 7.87 (d, J=8.8 Hz, 3H), 7.75 (d, J=8 Hz, 3H), 7.62 (d, J=8.4 Hz, 4H), 7.39 (m, 3H), 2.44 (m, 1H), 1.79 (m, 1H), 1.54 (m, 8H), 0.90 (m, 5H). EIMS m/z M⁺¹ 497.6. Anal. (C, H, N)

N-(4-(5-(4-(4-methylcyclohexanecarboxamido)phenyl)-1H-imidazol-2-yl)phenyl)-4-chlorobenzamide. Reverse phase chromatography over C18 using H₂O/ACN/TFA as eluent to yield the product as a tan solid (196 mg, 0.382 mmol, 47%) Mp: 317-318° C. ¹H NMR (400 MHz, DMSO-d₆) δ9.80 (m, 1H), 8.00 (m, 4H), 7.86 (d, J=8.8 Hz, 4H), 7.75 (d, J=8.4 Hz, 2H), 7.62 (m, 5H), 2.44 (m, 1H), 1.77 (m, 3H), 1.52 (m, 6H), 0.90 (m, 4H). EIMS m/z M⁺¹ 513.4. Anal. (C, H, N)

N-(4-(5-(4-(4-methylcyclohexanecarboxamido)phenyl)-1H-imidazol-2-yl)phenyl)-3,4-dichlorobenzamide. Reverse phase chromatography over C18 using H₂O/ACN/TFA as eluent to yield the product as a tan solid (160 mg, 0.292 mmol, 36%) Mp: 274-275° C. ¹H NMR (400 MHz, DMSO-d₆) δ12.51 (bs, 1H), 10.51 (s, 1H), 9.80 (apparent d, 1H), 8.24 (d, J=2 Hz, 1H), 7.98 (m, 2H), 7.85 (m, 3H), 7.75 (d, J=8 Hz, 2H), 7.63 (d, J=8.4 Hz, 3H), 2.44 (m, 1H), 1.78 (m, 4H), 1.51 (m, 6H), 0.92 (m, 4H). EIMS m/z M⁺¹ 547.6. Anal. (C, H, N)

N-(4-(5-(4-(4-methylcyclohexanecarboxamido)phenyl)-1H-imidazol-2-yl)phenyl)-4-methoxybenzamide. Reverse phase chromatography over C18 using H₂O/ACN/TFA as eluent to yield the product as a tan solid (160 mg, 0.315 mmol, 39%) Mp: 285-286° C. ¹H NMR (400 MHz, DMSO-d₆) δ10.20 (bs, 1H), 9.80 (m, 1H), 9.97 (m, 5H), 7.87 (d, J=8.8 Hz, 2H), 7.75 (d, J=8.4 Hz, 3H), 7.08 (m, 2H), 3.85 (s, 3H), 2.44 (m, 1H), 1.79 (m, 4H), 1.52 (m, 6H), 0.92 (m, 4H). EIMS m/z M⁺¹ 509.6. Anal. (C, H, N)

N-(4-(5-(4-(4-methylcyclohexanecarboxamido)phenyl)-1H-imidazol-2-yl)phenyl)-2,3,4,5,6-pentafluorobenzamide. Reverse phase chromatography over C18 using H₂O/ACN/TFA as eluent to yield the product as a tan solid (47 mg, 0.085 mmol, 8%) Mp: 295° C. ¹H NMR (400 MHz, DMSO-d₆) δ11.25 (s, 1H), 9.88 (s, 1H), 8.06 (d, J=8.8 Hz, 2H), 7.78 (m, 5H), 7.67 (d, J=8.4 Hz, 2H), 2.34 (m, 1H), 1.79 (m, 4H), 1.65 (m, 1H), 1.42 (m, 2H), 1.23 (m, 4H), 0.92 (m, 4H). EIMS m/z M⁺¹ 555.4. Anal. (C, H, N)

N-(4-(2-(4-Adamatylamidophenyl)-1H-imidazol-5-yl)phenyl)cycloheptanecarboxamide: R_{f} (95:5 CH₂Cl₂-MeOH) 0.26; mp 212-4° C. ¹H NMR (DMSO-d₆, δ in ppm): 9.78 (s, 1 H), 9.24 (s, 1 H), 7.92 (d, J=8.4 Hz, 2 H), 7.78 (br. d, J=8.8 Hz, 4 H), 7.63 (br. s, 1 H), 7.62 (d, J=8.8 Hz, 2 H), 2.56-1.47 (m, 28 H). MS: [EI] m/e 537.6 [M+H]⁺. Anal: (C₃₄H₄₀N₄O₂-0.92 H₂O) C, H, N.

N-(4-(2-(4-(Cyclohexanecarboxamido)phenyl)-1H-imidazol-5-yl)phenyl)cycloheptanecarboxamide: R_{f} (95:5 CH₂Cl₂-MeOH) 0.17; mp 192-4° C. ¹H NMR (DMSO-d₆, δ in ppm): 10.08 (s, 1 H), 9.72 (s, 1 H), 7.99 (d, J=8.8 Hz, 2 H), 7.82-7.79 (m, 4 H), 7.80 (br. s, 1 H), 7.71 (d, J=8.4 Hz, 2 H), 2.52-1.23 (m, 24 H). MS: [EI] m/e 485.4 [M+H]⁺. Anal: (C₃₀H₃₆N₄O₂-2.67 H₂O) C, H, N.

N-(4-(2-(4-(2-Methylcyclohexanecarboxamido)phenyl)-1H-imidazol-5-yl)phenyl)cycloheptanecarboxamide: R_{f} (95:5 CH₂Cl₂-MeOH) 0.19; mp 258-60° C. A mixture of diastereomers in 83:17 ratio. ¹H NMR (DMSO-d₆, δ in ppm): 9.91 (s, 1 H), 9.82 (s, 1 H), 7.91 (d, J=8.8 Hz, 2 H), 7.74 (d, J=8.8 Hz, 2 H), 7.70 (d, J=8.8 Hz), 2 H), 7.64 (s, 1 H), 7.63 (d, J=8.4 Hz, 2 H), 2.57-1.23 (m, 23 H), 0.89 (d, J=6.8 Hz, 2.5 H), 0.84 (d, J=6.4 Hz, 0.5 H). MS: [EI] m/e 499.4 [M+H]⁺. Anal: (C₃₁H₃₈N₄O₂-1.76 H₂O) C, H, N.

N-(4-(2-(4-(4-Methylcyclohexanecarboxamido)phenyl)-1H-imidazol-5-yl)phenyl)cycloheptanecarboxamide: R_{f} (95:5 CH₂Cl₂-MeOH) 0.19; mp 244-6° C. A mixture of diastereomers in 86:14 ratio. ¹H NMR (DMSO-d₆, δ in ppm): 10.04 (s, 1 H), 9.91 (s, 1 H), 7.95 (d, J=8.8 Hz, 2 H), 7.86 (s, 1 H), 7.79 (d, J=8.4 Hz, 2 H), 7.77 (d, J=8.4 Hz, 2 H), 7.68 (d, J=8.8 Hz, 2 H), 2.56-1.44 (m, 23 H), 0.93 (d, J=7.2 Hz, 2.6 H), 0.89 (d, J=6.4 Hz, 0.4 H). MS: [EI] m/e 499.4 [M+H]⁺. Anal: (C₃₁H₃₈N₄O₂-3.64 H₂O-0.05 CF₃CO₂H) C, H, N.

N-(4-(5-(4-(Cycloheptanecarboxamido)phenyl)-1H-imidazol-2-yl)phenyl)nicotinamide: R_{f} (95:5 CH₂Cl₂-MeOH) 0.04; mp 326-8° C. ¹H NMR (DMSO-d₆, δ in ppm): 10.70 (s, 1 H), 9.91 (s, 1 H), 9.14 (d, J=2.0 Hz, 1 H), 8.79 (dd, J=5.0, 2.0 Hz, 1 H), 8.32 (td, J=8.0, 2.0 Hz, 1 H), 8.04 (d, J=8.8 Hz, 2 H), 7.97 (d, J=8.8 Hz, 2 H), 7.89 (s, 1 H), 7.79 (d, J=8.8 Hz, 2 H), 7.69 (d, J=8.8 Hz, 2 H), 7.61 (ddd, J=8.0, 5.0, 1.0 Hz, 1 H), 2.54-1.67 (m, 13 H). MS: [EI] m/e 480.4 [M+H]⁺. Anal: (C₂₉H₂₉N₅O₂-3.17 H₂O-0.10 CF₃CO₂H) C, H, N.

N-(4-(2-(4-(Benzamido)phenyl)-1H-imidazol-5-yl)sphenyl)cycloheptanecarboxamide: R_{f} (95:5 CH₂Cl₂-MeOH) 0.20; mp 310-2° C. ¹H NMR (DMSO-d₆, δ in ppm): 10.65 (s, 1 H), 10.06 (s, 1 H), 8.20 (d, J=8.8 Hz, 2 H), 8.19-8.16 (m, 2 H), 8.14 (d, J=9.2 Hz, 2 H), 7.98-7.96 (m, 2 H), 7.96 (s, 1 H), 7.86 (d, J=8.8 Hz, 2 H), 7.83-7.73 (m, 3 H), 2.72-1.64 (m, 13 H). MS: [EI] m/e 479.4 [M+H]³⁰. Anal: (C₃₀H₃₀N₄O₂-2.0 H₂O) C, H, N.

N-(4-(2-(4-(2,3,4,5,6-Pentafluorobenzamido)phenyl)-1H-imidazol-5-yl)phenyl)cycloheptanecarboxamide: R_{f} (95:5 CH₂Cl₂-MeOH) 0.27; mp 300-2° C. ¹H NMR (DMSO-d₆, δ in ppm): 11.33 (s, 1 H), 9.94 (s, 1 H), 8.08 (d, J=8.8 Hz, 2 H), 7.98 (s, 1 H), 7.87 (d, J=8.8 Hz, 2 H), 7.80 (d, J=8.8 Hz, 2 H), 7.72 (d, J=8.4 Hz, 2 H), 2.54-1.45 (m, 13 H). MS: [EI] m/e 569.4 [M+H]⁺. Anal: (C₃₀H₂₅F₅N₄O₂-3.26 H₂O-0.14 CF₃CO₂H) C, H, N.

N-(4-(2-(4-(3,4-Dichlorobenzamido)phenyl)-1H-imidazol-5-yl)phenyl)cycloheptanecarboxamide: R_{f} (95:5 CH₂Cl₂-MeOH) 0.21; mp 304-6° C. ¹H NMR (DMSO-d₆, δ in ppm): 10.63 (s, 1 H), 9.91 (s, 1 H), 8.23 (s, 1 H), 8.03 (d, J=8.0 Hz, 2 H), 7.96 (s, 1 H), 7.95 (d, J=8.4 Hz, 2 H), 7.95-7.91 (m, 1 H), 7.84 (d, J=8.0 Hz, 1 H), 7.80 (d, J=8.0 Hz, 2 H), 7.68 (d, J=8.0 Hz, 2 H), 2.54-1.46 (m, 13 H). MS: [EI] m/e 547.6 [M+H]⁺. Anal: (C₃₀H₂₈Cl₂N₄O₂-3.49 H₂O) C, H, N.

N-(4-(2-(4-(4-Fluorobenzamido)phenyl)-1H-imidazol-5-yl)phenyl)cycloheptanecarboxamide: R_{f} (95:5 CH₂Cl₂-MeOH) 0.24; mp 314-6° C. ¹H NMR (DMSO-d₆, δ in ppm): 10.54 (s, 1 H), 9.94 (s, 1 H), 8.07 (ddd, J=8.8, 5.6, 2.0 Hz, 2 H), 8.04 (d, J=9.2 Hz, 2 H), 8.03 (s, 1 H), 7.99 (d, J=9.2 Hz, 2 H), 7.80 (d, J=8.8 Hz, 2 H), 7.71 (d, J=8.8 Hz, 2 H), 7.40 (dt, J=8.8, 2.0 Hz, 2 H), 2.54-1.45 (m, 13 H). MS: [EI] m/e 497.6 [M+H]⁺. Anal: (C₃₀H₂₉FN₄O₂-3.58 H₂O-0.04 CF₃CO₂H) C, H, N.

N-(4-(2-(4-(4-Chlorobenzamido)phenyl)-1H-imidazol-5-yl)phenyl)cycloheptanecarboxamide: R_{f} (95:5 CH₂Cl₂-MeOH) 0.23; mp 325-7° C. ¹H NMR (DMSO-d₆, δ in ppm): 10.61 (s, 1 H), 9.96 (s, 1 H), 8.06-7.99 (m, 7 H), 7.80 (d, J=8.8 Hz, 2 H), 7.72 (d, J=8.8 Hz, 2 H), 7.64 (dd, J=8.8, 2.0 Hz, 2 H), 2.54-1.45 (m, 13 H). MS: [EI] m/e 513.4 [M+H]⁺. Anal: (C₃₀H₂₉ClN₄O₂-3.39 H₂O-0.22 CF₃CO₂H) C, H, N.

N-(4-(2-(4-(4-Methoxybenzamido)phenyl)-1H-imidazol-5-yl)phenyl)cycloheptanecarboxamide: R_{f} (95:5 CH₂Cl₂-MeOH) 0.23; mp 311-3° C. ¹H NMR (DMSO-d₆, δ in ppm): 10.30 (s, 1 H), 9.89 (s, 1 H), 8.06 (d, J=8.8 Hz, 2 H), 8.04 (d, J=8.4 Hz, 2 H), 7.96 (d, J=8.8 Hz, 2 H), 7.83 (d, J=8.4 Hz, 2 H), 7.11 (s, 1 H), 7.70 (d, J=8.8 Hz, 2 H), 7.15 (d, J=8.8 Hz, 2 H), 2.56-1.52 (m, 13 H). MS: [EI] m/e 509.6 [M+H]⁺. Anal: (C₃₁H₃₂N₄O₃-2.77 H₂O) C, H, N.

N-(4-(2-(4-(4-Nitrobenzamido)phenyl)-1H-imidazol-5-yl)phenyl)cycloheptanecarboxamide: R_{f} (95:5 CH₂Cl₂-MeOH) 0.20; mp 236-8° C. ¹H NMR (DMSO-d₆, δ in ppm): 10.30 (s, 1 H), 9.89 (s, 1 H), 8.41 (d, J=9.2 Hz, 2 H), 8.22 (d, J=9.2 Hz, 2 H), 8.07 (d, J=9.2 Hz, 2 H), 8.03 (s, 1 H), 8.02 (d, J=8.4 Hz, 2 H), 7.83 (d, J=8.8 Hz, 2 H), 7.75 (d, J=8.8 Hz, 2 H), 2.56-1.43 (m, 13 H). MS: [EI] m/e 524.4 [M+H]³⁰. Anal: (C₃₀H₂₉N₅O₄-4.38 H₂O-0.28 CF₃CO₂H) C, H, N.

N-(4-(2-(4-(1-Adamantanecarboxamido)phenyl)-1H-imidazol-5-yl)phenyl)nicotinamide: R_{f} (90:10 CH₂Cl₂-MeOH) 0.41; mp 251-2° C. ¹H NMR (DMSO-d₆, δ in ppm): 10.64 (s, 1 H), 9.49 (s, 1 H), 9.13 (d, J=1.6 Hz, 1 H), 8.79 (dd, J=4.8, 1.6 Hz, 1 H), 8.32 (td, J=8.0, 1.6 Hz, 1 H), 8.11 (s, 1 H), 8.01 (d, J=8.8 Hz, 2 H), 7.95 (d, J=8.8 Hz, 2 H), 7.94 (d, J=8.8 Hz, 2 H), 7.90 (d, J=8.8 Hz, 2 H), 7.60 (ddd, J=8.0, 4.8, 0.4 Hz, 1 H), 2.04 (br. s, 3 H), 1.94 (br. S, 6 H), 1.72 (br. S, 6 H). MS: [EI] m/e 518.4 [M+H]⁺. Anal: (C₃₂H₃₁N₅O₂-1.43 H₂O-0.98 CF₃CO₂H) C, H, N.

N-(4-(2-(4-(Cyclohexanecarboxamido)phenyl)-1H-imidazol-5-yl)phenyl)nicotinamide: R_{f} (90:10 CH₂Cl₂-MeOH) 0.31; mp 315-7° C. ¹H NMR (DMSO-d₆, δ in ppm): 10.61 (s, 1 H), 10.18 (s, 1 H), 9.13 (d, J=1.6 Hz, 1 H), 8.79 (dd, J=4.8, 1.6 Hz, 1 H), 8.32 (td, J=8.0, 1.6 Hz, 1 H), 8.07 (s, 1 H), 7.99 (d, J=8.8 Hz, 2 H), 7.93 (d, J=8.8 Hz, 2 H), 7.89 (d, J=8.8 Hz, 2 H), 7.84 (d, J=8.8 Hz, 2 H), 7.61 (ddd, J=8.0, 4.8, 0.8 Hz, 1 H), 2.38-1.18 (m, 11 H). MS: [EI] m/e 466.6 [M+H]⁺. Anal: (C₂₈H₂₇N₅O₂-2.17 H₂O-0.99 CF₃CO₂H) C, H, N.

N-(4-(2-(4-(2-Methylcyclohexanecarboxamido)phenyl)-1H-imidazol-5-yl)phenyl)nicotinamide: R_{f} (90:10 CH₂Cl₂-MeOH) 0.34; mp 245-7° C. A mixture of diastereomers in 83:17 ratio. ¹H NMR (DMSO-d₆, δ in ppm): 10.63 (s, 1 H), 10.15 (s, 1 H), 9.13 (dd, J=2.2, 0.6 Hz, 1 H), 8.79 (dd, J=4.8, 1.6 Hz, 1 H), 8.32 (td, J=8.0, 2.0 Hz, 1 H), 8.09 (s, 1 H), 8.00 (d, J=8.8 Hz, 2 H), 7.93 (d, J=8.8 Hz, 2 H), 7.89 (d, J=8.8 Hz, 2 H), 7.84 (d, J=8.8 Hz, 2 H), 7.61 (ddd, J=8.0, 4.8, 0.8 Hz, 1 H), 2.61-1.27 (m, 10 H), 0.89 (d, J=6.8 Hz, 2.5 H), 0.85 (d, J=6.4 Hz, 0.5 H). MS: [EI] m/e 480.4 [M+H]⁺. Anal: (C₂₉H₂₉N₅O₂-2.54 H₂O-0.75 CF₃CO₂H) C, H, N.

N-(4-(2-(4-(4-Methylcyclohexanecarboxamido)phenyl)-1H-imidazol-5-yl)phenyl)nicotineamide: R_{f} (90:10 CH₂Cl₂-MeOH) 0.32; mp 230-2° C. A mixture of diastereomers in 86:14 ratio. ¹H NMR (DMSO-d₆, δ in ppm): 10.63 (s, 1 H), 10.15 (s, 1 H), 9.13 (dd, J=2.2, 0.6 Hz, 1 H), 8.79 (dd, J=4.8, 1.6 Hz, 1 H), 8.32 (td, J=8.0, 2.0 Hz, 1 H), 8.11 (s, 1 H), 8.01 (dd, J=8.8, 1.6 Hz, 2 H), 7.94 (d, J=8.8 Hz, 2 H), 7.90 (d, J=8.8 Hz, 2 H), 7.85 (dd, J=8.8, 2.4 Hz, 2 H), 7.61 (ddd, J=8.0, 4.8, 0.8 Hz, 1 H), 2.61-1.27 (m, 10 H), 0.94 (d, J=7.2 Hz, 2.6 H), 0.89 (d, J=6.8 Hz, 0.4 H). MS: [EI] m/e 480.4 [M+H]⁺. Anal: (C₂₉H₂₉N₅O₂-1.90 H₂O-0.71 CF₃CO₂H) C, H, N.

N-(4-(2-(4-(Nicotinamido)phenyl)-1H-imidazol-5-yl)phenyl)nicotinamide: R_{f} (90:10 CH₂Cl₂-MeOH) 0.14; mp 317-9° C. ¹H NMR (DMSO-d₆, δ in ppm): 10.87 (s, 1 H), 10.71 (s, 1 H), 9.17 (s, 2 H), 8.83-8.81 (m, 2 H), 8.39 (br. d, J=8.0 Hz, 2 H), 8.20 (s, 1 H), 8.12 (dd, J=8.8, 1.6 Hz, 2 H), 8.06 (d, J=9.2 Hz, 2 H), 7.96 (d, J=9.2 Hz, 2 H), 7.93 (d, J=8.8 Hz, 2 H), 7.65 (dd, J=8.0, 4.8 Hz, 1 H). MS: [EI] m/e 461.4 [M+H]⁺. Anal: (C₂₇H₂₀N₆O₂-2.95 H₂O-2.38 CF₃CO₂H) C, H, N.

N-(4-(2-(4-(3,4-Dichlorobenzamido)phenyl)-1H-imidazol-5-yl)pehnyl)nicotinamide: R_{f} (90:10 CH₂Cl₂-MeOH) 0.34; mp 332-4° C. ¹H NMR (DMSO-d₆, δ in ppm): 10.74 (s, 1 H), 10.66 (s, 1 H), 9.13 (d, J=1.6 Hz, 1 H), 8.78 (dd, J=4.8, 1.6 Hz, 1 H), 8.32 (td, J=8.0, 2.0 Hz, 1 H), 8.23 (d, J=2.0 Hz, 1 H), 8.12 (s, 1 H), 8.08 (dd, J=8.8, 1.6 Hz, 2 H), 8.01 (d, J=9.2 Hz, 2 H), 7.95 (dd, J=8.4, 2.0 Hz, 1 H), 7.93 (d, J=8.4 Hz, 2 H), 7.90 (d, J=9.2 Hz, 2 H), 7.84 (d, J=8.4 Hz, 1 H), 7.60 (ddd, J=8.0, 4.8, 0.8 Hz, 1 H). MS: [EI] m/e 528.2 [M+H]⁺. Anal: (C₂₈H₁₉Cl₂N₅O₂-2.84 H₂O-0.60 CF₃CO₂H) C, H, N.

N-(4-(2-(4-(2,3,4,5,6-Pentafluorobenzamido)phenyl)-1H-imidazol-5-yl)phenyl)nicotinamide: R_{f} (90:10 CH₂Cl₂-MeOH) 0.37; mp 265-6° C. ¹H NMR(DMSO-d₆, δ in ppm): 11.35 (s, 1 H), 10.61 (s, 1 H), 9.13 (d, J=1.6 Hz, 1 H), 8.79 (dd, J=4.8, 1.6 Hz, 1H), 8.31 (td, J=8.0, 2.0 Hz, 1 H), 8.10 (d, J=8.8 Hz, 2 H), 8.09 (s, 1 H), 7.94-7.88 (m, 6 H), 7.61 (ddd, J=8.0, 4.8, 0.8 Hz, 1 H). MS: [EI] m/e 550.4 [M+H]⁺. Anal: (C₂₈H₁₆F₅N₅O₂-1.48 H₂O-1.02 CF₃CO₂H) C, H, N.

N-(4-(2-(4-(Cycloheptanecarboxamido)phenyl)-1H-imidazol-5-yl)phenyl)nicotinamide: R_{f} (90:10 CH₂Cl₂-MeOH) 0.39; mp 256-8° C. ¹H NMR (DMSO-d₆, δ in ppm): 10.47 (s, 1 H), 9.94 (s, 1 H), 9.12 (d, J=2.0 Hz, 1 H), 8.76 (dd, J=5.0, 2.0 Hz, 1 H), 8.31 (td, J=8.0, 2.0 Hz, 1 H), 7.92 (d, J=8.8 Hz, 2 H), 7.84 (d, J=8.8 Hz, 2 H), 7.81 (d, J=8.8 Hz, 2 H), 7.70 (d, J=8.8 Hz, 2 H), 7.68 (s, 1 H), 7.58 (ddd, J=8.0, 5.0, 1.0 Hz, 1 H), 2.54-1.45 (m, 13 H). MS: [EI] m/e 480.4 [M+H]⁺. Anal: (C₂₉H₂₉N₅O₂-0.42 H₂O-0.27 CF₃CO₂H) C, H, N.

2-Methyl-N-(4-(2-(4-(cyclohexanecarboxamido)phenyl)-1H-imidazol-5-yl)phenyl)cyclohexanecarboxamide: R_{f} (90:10 CH₂Cl₂-MeOH) 0.37; mp 221-3° C. A mixture of diastereomers in 83:17 ratio. ¹H NMR (DMSO-d₆, δ in ppm): 10.18 (s, 1 H), 9.93 (s, 1 H), 8.02 (s, 1 H), 7.98 (d, J=8.8 Hz, 2 H), 7.83 (d, J=8.8 Hz, 2 H), 7.79 (d, J=8.8 Hz, 2 H), 7.73 (d, J=8.8 Hz, 2 H), 2.54-1.18 (m, 21 H), 0.89 (d, J=6.8 Hz, 2.5 H), 0.84 (d, J=6.4 Hz, 0.5 H). MS: [EI] m/e 485.4 [M+H]⁺. Anal: (C₃₀H₃₆N₄O₂-1.61 H₂O-0.76 CF₃CO₂H) C, H, N.

N-(4-(5-(4-(2-methylcyclohexanecarboxamido)phenyl)-1H-imidazol-2-yl)phenyl)nicotinamide: R_{f} (90:10 CH₂Cl₂-MeOH) 0.20; mp 226-8° C. A mixture of diastereomers in 83:17 ratio. ¹H NMR (DMSO-d₆, δ in ppm): 10.81 (s, 1 H), 9.97 (s, 1 H), 9.15 (d, J=2.0 Hz, 1 H), 8.81 (dd, J=4.8, 1.6 Hz, 1 H), 8.34 (td, J=8.0, 2.0 Hz, 1 H), 8.13 (s, 1 H), 8.09 (d, J=9.2 Hz, 2 H), 8.05 (d, J=9.2 Hz, 2 H), 7.83 (d, J=9.2 Hz, 2 H), 7.76 (d, J=8.8 Hz, 2 H), 7.62 (ddd, J=8.0, 4.8, 0.8 Hz, 1 H), 2.58-1.27 (m, 10 H), 0.89 (d, J=6.8 Hz, 2.5 H), 0.85 (d, J=6.4 Hz, 0.5 H). MS: [(+) EI] m/e 480.4 [M+H]⁺. Anal: (C₂₉H₂₉N₅O₂-2.42 H₂O-1.98 CF₃CO₂H) C, H, N.

2-Methyl-N-(4-(2-(4-(4-methylcyclohexanamido)phenyl)-1H-imidazol-5-yl)phenyl)cyclohexanecarboxamide: R_{f} (92:8 CH₂Cl₂-MeOH) 0.36; mp 218-20° C. Two sets of amide protons are observed, on the top of a mixture of four possible diastereomers. ¹H NMR (DMSO-d₆, δ in ppm): 10.20 (s, 0.2 H), 10.14 (s, 0.8 H), 10.04 (s, 0.15 H), 9.94 (s, 0.85 H), 8.02 (s, 1 H), 7.98 (d, J=8.8 Hz, 2 H), 7.84 (d, J=8.8 Hz, 2 H), 7.79 (d, J=8.8 Hz, 2 H), 7.73 (d, J=8.8 Hz, 2 H), 2.54-1.31 (m, 20 H), 0.95-0.82 (m, 6 H). MS: [(+) EI] m/e 499.4 [M+H]⁺. Anal: (C₃₁H₃₈N₄O₂-1.74 H₂O-0.52 CF₃CO₂H) C, H, N.

N-(4-(5-(4-(2-Methylcyclohexanecarboxamido)phenyl)-1H-imidazol-2-yl)phenyl)cycloheptanecarboxamide: R_{f} (92:8 CH₂Cl₂-MeOH) 0.35; mp 220-2° C. A mixture of diastereomers in 83:17 ratio. ¹H NMR (DMSO-d₆, δ in ppm): 10.18 (s, 1 H), 10.05 (s, 0.15 H), 9.95 (s, 0.85 H), 8.03 (s, 1 H), 7.99 (d, J=8.8 Hz, 2 H), 7.83 (d, J=8.8 Hz, 2 H), 7.80 (d, J=8.8 Hz, 2 H), 7.73 (d, J=8.4 Hz, 2 H), 2.57-1.23 (m, 23 H), 0.89 (d, J=7.2 Hz, 2.5 H), 0.85 (d, J=6.4 Hz, 0.5 H). MS: [(+) EI] m/e 499.4 [M+H]⁺. Anal: (C₃₁H₃₈N₄O₂-1.21 H₂O-0.67 CF₃CO₂H) C, H, N.

N-(4-(5-(4-(2-Methylcyclohexanecarboxamido)phenyl)-1H-imidazol-2-yl)phenyl)picolinamide: R_{f} (92:8 CH₂Cl₂-MeOH) 0.30; mp 227-9° C. A mixture of diastereomers in 83:17 ratio. ¹H NMR (DMSO-d₆, δ in ppm): 11.00 (s, 1 H), 10.08 (s, 0.15 H), 9.97 (s, 0.85 H), 8.77 (d, J=4.8 Hz, 1 H), 8.20 (dd, J=8.0, 0.8 Hz, 1 H), 8.19 (d, J=8.8 Hz, 2 H), 8.11 (dd, J=8.0, 1.6 Hz, 1 H), 8.08 (s, 1 H), 8.07 (d, J=8.8 Hz, 2 H) 7.82 (d, J=8.8 Hz, 2 H), 7.34 (d, J=8.8 Hz, 2 H), 7.34-7.72 (m, 1 H), 2.57-1.23 (m, 10 H), 0.89 (d, J=7.2 Hz, 2.5 H), 0.85 (d, J=6.4 Hz, 0.5 H). MS: [EI] m/e 480.4 [M+H]⁺. Anal: (C₂₉H₂₉N₅O₂-1.62 H₂O-0.57 CF₃CO₂H) C, H, N.

### Synthetic Scheme 11

4-Nitrobenzamidine HCl (32). (prepared by the known method Journal of Organic Chemistry 55, 7, 1990, 2005-2004) To a solution of 4-nitrobenzonitrile (25.5 g, 172 mmol) in dry methanol (230 ml) was added a solution of sodium methoxide (1 g, 18.5 mmol) and the solution warmed until complete dissolution of the solid. The solution was stirred at room temperature for 55 h at which time solid NH₄Cl (9.5 g, 177 mmol) was added and the mixture heated at 45° C. for 48 h. The mixture was cooled to room temperature and the resulting solid collected by filtration, rinsed with acetone and dried to give the product as a yellow solid (21.6 g, 107 mmol, 62%). The crude product was used as is in subsequent steps.

4-(2-(4-nitrophenyl)-1H-imidazol-5-yl)benzonitrile (114). To a solution of 4-nitrobenzamidine (22) (2 g, 10 mmol) in THF (17 mL) and H₂O (5 mL) was added solid NaHCO₃ (3.36 g, 40 mmol) and the mixture brought to reflux. A solution of 4-cyanophenacyl bromide (113) (2.24 g, 10 mmol) in dry THF (4 mL) was added to the vigorously refluxing solution dropwise and the solution refluxed for 3 h. The THF was removed under reduced pressure and the residue diluted with water, sonicated, and collected by filtration then dried to give the product as a brown solid (2.14 g) that was used as is without further purification.

4-(2-(4-nitrophenyl)-1H-imidazol-5-yl)benzamide (115). To a solution of 4-(2-(4-nitrophenyl)-1H-imidazol-5-yl)benzonitrile (45) in CH₃OH (100 mL) was added LiOH-H₂O (6×, 1.75 g, 41 mmol) followed by H₂O₂ (50% w/w, 3 mL) and the mixture heated at reflux for 5 h. The solution was cooled and the pH adjusted to ˜4 using 20% HCl (aq). The resulting solid was collected and dried to give the product as an orange solid 1.20 g. A second amount of product was collected (0.207 g) from the filtrate. The product was used as in without further purification.

Methyl 4-(2-(4-nitrophenyl)-1H-imidazol-5-yl)benzoate (116). To a solution of 4-(2-(4-nitrophenyl)-1H-imidazol-5-yl)benzamide (115) (1.415 g, 4.6 mmol) in dry CH₃OH (150 mL) was added conc. HCl (25 mL) and the solution heated at reflux for 1 d. During this time the solid dissolved. TLC in DCM/MeOH (95/5) showed no more starting material and a major spot at Rf=0.51. The methanol was removed under reduced pressure and the resulting solid collected by filtration, rinsed with H₂O, and dried to give the product as a solid (1.37 g) that was used as is without further purification.

4-(2-(4-nitrophenyl)-1H-imidazol-5-yl)benzoic acid (117). Procedure used in route A. To a solution of methyl 4-(2-(4-nitrophenyl)-1H-imidazol-5-yl)benzoate (116) (1.37)g, 4.6 mmol) in EtOH (150 mL) was added 10% aqueous NaOH (20 mL) and the mixture heated at reflux for 3.5 h. The mixture was diluted with H₂O (20 mL) and most of the EtOH removed under reduced pressure. The pH of the remaining purple mixture was adjusted to pH˜4 with aqueous 20% HCl and stirred for 5 min. The product was given as an orange yellow solid that was collected by filtration and dried to give 1.11 g that was used as is without further purification. The product gave a baseline spot on the TLC in DCM/MeOH (95/5).

A second more efficient procedure for the synthesis of 4-(2-(4-nitrophenyl)-1H-imidazol-5-yl)benzoic acid (117) was used in route B. To a solution of 4-(2-(4-nitrophenyl)-1H-imidazol-5-yl)benzonitrile (114) (4.89 g, 16.8 mmol) in 20% aqueous KOH (250 mL) was heated at reflux for 1.75 h. The purple solution was cooled slightly and neutralized with 20% aqueous HCl until a solid precipitated. The solid was collected by filtration and rinsed with water and then dried in vacuuo to give 5.871 g of slightly wet solid that was used as is.

4-(2-(4-nitrophenyl)-1H-imidazol-5-yl)-N-(pyridin-2-yl)benzamide (118). To a solution of 4-(2-(4-nitrophenyl)-1H-imidazol-5-yl)benzoic acid (48) (5 g, 16 mmol) in dry dichloromethane (50 mL) was added (COCl₂ (2 mL) and the mixture heated at 35° C. for 18 h. The solvents were removed under reduced pressure to give a yellow/white residue.

The residue was dissolved in dry pyridine (50 mL) and 2-aminopyridine (1.2 eq, 1.88 g, 20 mmol) was added and the mixture stirred at room temperature for 3 h and then poured into water. The resulting yellow solid was collected by filtration and dried to give 3.683 g that was used as is in the following steps.

4-(2-(4-adamantylamidophenyl)-1H-imidazol-5-yl)-N-(pyridin-2-yl)benzamide (119). To a solution of 4-(2-(4-nitrophenyl)-1H-imidazol-5-yl)-N-(pyridin-2-yl)benzamide (118) (2.1 g, 5.5 mmol) in MeOH (150 mL) was added Raney Nickel and the mixture vacuum purged using H₂ gas. The mixture was stirred under H₂ gas at 80° C. for 5 h and the catalyst filtered off through celite. The filtrate was concentrated to give the product (1.23 g, 3.5mmol).

The residue (one third) was dissolved in pyridine (5 mL) and 1-adamantancarbonyl chloride (1.1 eq, 252 mg, 1.27 mmol) added and the mixture stirred for 15 h. Water was added and the mixture stirred for 15 h. The resulting solid was collected by filtration and purified by HPLC (C18, ACN/TFA/H₂O) to give the product as a solid. (80 mg, 0.15 mmol, 13%) Mp: 292-293° C. ¹H NMR (400 MHz, DMSO-d₆) δ10.69 (bs, 1H), 9.24 (s, 1H), 8.39 (m, 1H), 8.21(d, J=8 Hz, 1H), 8.08(d, J=8 Hz, 2H), 7.97 (t, J=8.4 Hz, 17 hz, 4H), 7.85 (m, 2H), 7.78(d, J=9.2 Hz, 2H), 7.17(m, 1H), 2.03(bs, 3H), 1.93 (bs, 6H) 1.72 (bs, 6H) EIMS m/z M⁺¹ 518.4. Anal. (C, H, N)

The following compounds were made using the method described above.

N-(4-(5-(4-(pyridin-2-ylcarbamoyl)phenyl)-1H-imidazol-2-yl)phenyl)cycloheptanecarboxamide. Product as a white solid. (30 mg, 0.06 mmol, 5%) Mp: 290-291° C. ¹H NMR (400 MHz, DMSO-d₆) δ10.69 (bs, 1H), 9.92 (s, 1H), 8.21 d, J=8.4 Hz, 1H), 8.07 (d, J=8.4 Hz, 2H), 7.95(dd, J=1.2 Hz, 8.4 Hz, 4H), 7.84 (m, 2H), 7.69 (d, J=8.8 Hz, 2H), 7.16(m, 1H), 1.86 (m, 2H), 1.8-1.4(m, 11H). EIMS m/z M⁺¹ 480.4. Anal. (C, H,N)

N-(4-(5-(4-(pyridin-2-ylcarbamoyl)phenyl)-1H-imidazol-2-yl)phenyl)cyclohexanecarboxamide. Product as a yellow solid. (29 mg, 0.06 mmol, 5%) Mp: 287-290° C. ¹H NMR (400 MHz, DMSO-d₆) δ10.68 (bs, 1H), 9.97 (s, 1H), 8.39 m, 1H), 8.19 (d, J=8.4 Hz, 1H), 8.06 (d, J=8.4 Hz, 2H), 7.94 (dd, J=11.2 Hz, 2.4 hz, 2H), 7.84 (m, 2H), 7.70 (d, J=8.8 Hz, 2H), 7.16 (ddd, J=0.8 Hz, 2.4 Hz, 7.6 Hz, 1H), 2.34 (m, 1H), 1.78 (m, 4H), 1.65 (m, 1H), 1.49-1.10 (m, 5H). EIMS m/z M⁺¹ 466.6. Anal. (C, H, N+1TFA)

N-(4-(5-(4-(cycloheptylcarbamoyl)phenyl)-1H-imidazol-2-yl)phenyl)benzenamide. Product as a white solid.(65 mg, 0.14 mmol, 27%) Mp: 161° C. ¹H NMR (400 MHz, DMSO-d₆) δ10.58 (s, 1H), 8.31 (d, J=7.6 Hz, 1H), 8.23 (s, 1H), 8.03 (m, 11H), 7.59 (m, 3H), 3.97 (m, 2H), 1.87 (m, 2H), 1.56 (m, 11H). EIMS m/z M⁺¹ 479.4. Anal. (C, H, N+1TFA)

N-(4-(5-(4-(cycloheptylcarbamoyl)phenyl)-1H-imidazol-2-yl)phenyl)picolinamide. Product as a brown solid. (135 mg, 0.285 mmol, 52%) Mp: 80° C. ¹H NMR (400 MHz, DMSO-d₆) δ10.94 (bs, 1H), 9.97 (m, 1H), 8.29 (d, J=7.6 Hz, 1H), 8.19 (m, 4H), 8.09 (m, 4H), 7.97 (s, 4H), 7.72 (m, 1H), 3.97 (m, 1H), 1.86 (m, 2H) 1.61 (m, 12H). EIMS m/z M⁺¹ 480.4. Anal. (C, H, N+1TFA)

N-(4-(5-(4-(cycloheptylcarbamoyl)phenyl)-1H-imidazol-2-yl)phenyl)cycloheptanecarboxamide. Product as a white solid. (92 mg, 0.184 mmol, 26%) Mp: 80° C. ¹H NMR (400 MHz, DMSO-d₆) δ10.19 (s, 1H), 8.32 (d, J=8 Hz, 1H), 8.22 (s, 1H), 8.02 (m, 6H), 7.83 (d, J=8.8 Hz, 2H), 3.98 (m, 2H), 2.52 (m, 1H), 1.84 (m, 4H), 160 (m, 21H). EIMS m/z M⁺¹ 499.4. Anal. (C, H, N+2TFA)

4-(2-(4-(4-methylcyclohexanecarboxamido)phenyl)-1H-imidazol-5-yl)-N-cycloheptylbenzamide. Product as a white solid. (60 mg, 0.12 mmol, 19%) Mp: 231-232° C. ¹H NMR (400 MHz, DMSO-d₆) δ9.96 (apparent d, 1H), 8.21 (d, J=7.6 Hz, 1H), 7.90 m, 7H), 7.72 (m, 2H), 3.97 (m, 1H), 2.46 (m, 1H), 2.28 (m, 0.3H), 1.58 (m, 21H), 0.90 (m, 4H), 2.34 (m, 1H), 1.79 (m, 4H), 1.65 (m, 1H), 1.49-1.10 (m, 5H). EIMS m/z M⁺¹ 499.6. Anal. (C, H, N)

4-(2-(4-(2-methylcyclohexanecarboxamido)phenyl)-1H-imidazol-5-yl)-N-cycloheptylbenzamide. Product as a white solid. (15 mg, 0.03 mmol, 6%) Mp: 204° C. ¹H NMR (400 MHz, DMSO-d₆) δ10.14 (apparent d, 1H), 8.30 (d, J=7.6 Hz, 1H), 8.17 (s, 1H), 8.00 (m, 6H), 7.83 (m, 2H), 3.96 (m, 1H), 2.58 (m, 1H), 2.14 (bs, 1H), 1.84 (m, 2H), 1.52 (m, 19H), 0.87 (m, 3H). EIMS m/z M⁺¹ 499.6. Anal. (C, H, N)

4-(2-(4-adamantylamidophenyl)-1H-imidazol-5-yl)-N-cycloheptylbenzamide. Product as a white solid. (127 mg, 0.237 mmol, 38%) Mp: 232° C. ¹H NMR (400 MHz, DMSO-d₆) δ9.45 (s, 1H), 8.30 (d, J=7.6 Hz, 1H), 8.19 (s, 1H), 7.96(m, 8H), 3.98 (m, 1H), 2.04(bs, 3H), 2.0-1.35 (series of m, 27H). EIMS m/z M⁺¹ 537.6. Anal. (C, H, N)

Adamantane-1-carboxylic acid (4-{5-[4-(adamantan-2-ylcarbamoyl)-phenyl]-1H-imidazol-2-yl}-phenyl)-amide. Product as a white solid. (202 mg, 0.351 mmol, 35%) Mp: 249° C. ¹H NMR (400 MHz, DMSO-d₆) δ12.56 (apparent d, 1H), 9.22 (s, 1H), 7.91 (m, 8H), 7.78 (d, J=8.8 Hz, 2H), 4.04 (m, 1H), 2.14 (m, 2H), 2.01 (m, 6H), 1.93 (bs, 7H), 1.84 (m, 8H), 1.72 (m, 9H), 1.53 (m, 3H). EIMS m/z M⁺¹ 575.8. Anal. (C, H, N)

N-Adamantan-2-yl-4-[2-[4-(cyclohexanecarbonyl-amino)-phenyl]-3H-imidazol-4-yl}-benzamide. Product as a white solid. (59 mg, 0.113 mmol, 11%) Mp: 331° C. ¹H NMR (400 MHz, DMSO-d₆) δ12.59 (bs, 1H), 9.94 (s, 1H), 7.89 (m, 9H), 7.70 (d, J=8.4 Hz, 3H), 4.05 (m, 1H), 2.35 (m, 1H), 2.14 (m, 2H), 2.00 (bs, 2H), 1.79 (m, 14H), 1.66 (m, 1H), 1.53 (d, J=12 Hz, 2H), 1.42 (m, 2H), 1.26 (m, 4H). EIMS m/z M^{30 1} 523.6. Anal. (C, H,N)

Cycloheptane carboxylic acid (4-{5-[4-(adamantan-2-ylcarbamoyl)-phenyl]-1H-imidazol-2-yl}-phenyl)-amide. Product as a white solid. (231 mg, 0.430 mmol, 42%) Mp: 236° C. ¹H NMR (400 MHz, DMSO-d₆) δ12.58 (bs, 1H), 9.92 (s, 1H), 7.88 (m, 10H), 7.70 (d, J=8.4 Hz, 3H), 4.05 (m, 1H), 2.14 (m, 3H), 1.99 (bs, 3H), 1.63 (series of m, 30H). EIMS m/z M⁺¹ 537.6. Anal. (C, H, N)

Pyridine-2-carboxylic acid (4-{5-[4-(adamantan-2-ylcarbamoyl)-phenyl]-1H-imidazol-2yl}-phenyl)-amide. Product as a white solid. (50 mg, 0.97 mmol, 11%) Mp: 331° C. ¹H NMR (400 MHz, DMSO-d₆) δ12.59 (bs, 1H), 9.94 (s, 1H), 7.89 (m, 9H), 7.70 (m, 1H), 4.05 (m, 1H), 2.14 (d, J=12.4 Hz, 2H), 2.00 (bs, 2H), 1.84 (m, 7H), 1.73(s, 2H), 1.53 (d, J=12.4 Hz, 2H), 1.42 (m, 2H), 1.26 (m, 4H). EIMS m/z M⁺¹ 518.4.6. Anal. (C, H, N)

### Synthetic Scheme 12

Methyl 4-(bromoacetyl)benzoate (122): To a solution of methyl 4-acetyl benzoate (121) (5.0 g, 28 mmol) in glacial AcOH (25 mL), bromine (1.5 ml, 4.67 g, 29 mmol) was added over 12 min at <20° C. Towards the end of the addition, solids started to appear. After stirring for additional 1.5 h, the solids were filtered, washed first with 50% aq. EtOH (60 mL) to remove excess bromine (clear filtrate), then with water (20 mL). Upon drying the material, cream-colored solids were obtained (6.62 g, 91.8%). ¹H NMR indicated traces of dibromo-derivative were present. Without further purification the material was used in the next step.

Methyl 4-(2-(4-nitrophenyl)-1H-imidazol-5-yl)benzoate (123): To a mixture of the 4-nitrobenzamidine hydrochloride (32; 1.0 g, 4.96 mmol), and NaHCO₃ (1.67 g, 19.84 mmol), THF (20 mL) and water (5 mL) were added and heated to reflux for 10 min and reaction flask was removed from the bath momentarily and bromo-derivative 122 (1.28 g, 4.96 mmol) was added and washed down to the flask with THF (5 mL). The dark-brown mixture was kept at reflux for additional 2 h. The volatile materials were removed in a rotary evaporator. Water (20 mL) was added to the residue and the solids were filtered, washed with water (20 mL) and dried overnight in vacuum oven at 80° C. The imidazole 123, obtained as medium-brown solid (1.48 g, 91.9%), was used in the next step.

4-(2-(4-Nitrophenyl)-1H-imidazol-5-yl)benzoic acid (124): The ester 123 (24.0 g, 0.074 mol) was taken up in 1:1 mixture of THF-MeOH (200 mL). Aq. 10% NaOH (156 mL, 0.15 mol) was added and heated at 60° C. overnight. After the volatiles were removed in a rotary evaporator, the residue was acidified with aq. 5 M HCl (pH˜4). The solids were filtered, washed with water (100 mL) and dried in vacuum oven at 80° C. to obtain desired acid 54 as brown solid (22.5 g, 98%).

N-Cyclohexyl-4-(2-(4-nitrophenyl)-1H-imidazol-5-yl)benzamide (125): To a suspension of the acid 124 (3.5 g, 11.3 mmol) in 1,2-dichloroethane (25 mL), thionyl chloride (1.24 mL, 2.02 g, 17.0 mmol) was added, followed by catalytic amount of DMF (3 drops) under argon. After heating at 80° C. for 24 h, the volatiles were removed in a rotary evaporator and dried under vacuum to obtain corresponding acid chloride hydrochloride salt. It was used immediately in the next step.

The acid chloride hydrochloride salt was added to a solution of cyclohexyl amine (1.35 g, 13.6 mmol) in pyridine (20 mL). After stirred for 16 h, the solvent was removed and the residue was treated with aq. NaHCO₃ (25 mL). The slurry was filtered, washed with water (25 mL) and dried to yield amide 125 as brown solids (3.21 g, 72.6%).

4-(2-(4-Aminophenyl)-1H-imidazol-5-yl)-N-cyclohexylbenzamide (126): The nitro compound 125 (1.2 g, 3.07 mmol) was taken up in 4:1 mixture of MeOH-THF (75 mL). The system was purged with argon, then with hydrogen (from balloon). Raney-Ni (slurry in water, 1.0 mL) was added and heated at 42° C. for 16 h. After cooling to rt, the reaction mixture was filtered through a pad of Celite, and washed with MeOH (50 mL). The filtrates were evaporated and dried to obtain amine 126 as brown mass (1.1 g, 99.2%).

N-Cyclohexyl-4-(2-(4-(1-adamantanamido)phenyl)-1H-imidazol-5-yl)benzamide (127): 1-Adamantane carbonyl chloride (0.19 g, 0.98 mmol) was added to a solution of the amine 126 (0.22 g, 0.61 mmol) in pyridine (5 mL) and stirred at rt for 15 h. After removal of the solvent, the residue was treated with aq. NaHCO₃ to obtain slurry. The solids were filtered, washed with water (25 mL), and dried to obtain the desired crude amide 127. The product was purified by reverse-phase chromatography (Combiflash; solvent system: CH₃CN/H₂O). The pure fractions were combined and evaporated off the volatiles (mostly the CH₃CN). Then sat'd NaHCO₃ (5 mL) was added and solids started to precipitate. The solids were filtered, washed with water (2×10 mL) and dried in vacuum oven at 80° C. overnight to obtain off-white solid (0.175 g, 54.9%); mp 247-9° C. ¹H NMR (DMSO-d₆, δ in ppm): 9.30 (s, 1 H), 8.21 (d, J=8.0 Hz, 1 H), 8.02-7.90 (m, 4 H), 7.97 (d, J=8.0, Hz, 2 H), 7.93 (s, 1 H), 7.85 (d, J=8.4. Hz, 2 H), 2.57-1.32 (m, 11 H), 2.04 (br. s, 3 H), 1.93 (br. s, 6 H), 1.72 (br. s, 6 H). MS: [EI] m/e 523.6 [M+H]⁺. Anal: (C₃₃H₃₈N₄O₂-2.86 H₂O-1.0 CF₃CO₂H) C, H, N.

The following compounds were prepared using above route.

N-(4-(5-(4-(Cyclohexylcarbamoyl)phenyl)-1H-imidazol-2-yl)phenyl)picolinamide: mp 288-90° C. ¹H NMR (DMSO-d₆, δ in ppm): 10.76 (s, 1 H), 8.76 (d, J=4.4 Hz, 1 H), 8.18 (d, J=7.6 Hz, 1 H), 8.14 (d, J=8.0 Hz, 1 H), 8.09 (dt, J=7.6, 0.8 Hz, 1 H), 8.04 (d, J=8.8 Hz, 2 H), 8.01 (d, J=9.2 Hz, 2 H), 7.91 (d, J=8.4 Hz, 2 H), 7.88 (s, 1 H), 7.87 (d, J=8.4 Hz, 2 H), 7.70 (dd, J=7.6, 4.8 Hz, 1 H), 3.78-3.75 (m, 1 H), 1.82 (br. s, 2 H), 1.75 (br. s, 2 H), 1.61 (br. d, J=12.0 Hz, 1 H), 1.32 (br. s, 4 H), 1.15 (br. t, J=8.4 Hz, 1 H). MS: [EI] m/e 466.6 [M+H]⁺. Anal: (C₂₈H₂₇N₅O₂-3.22 H₂O-0.24 CF₃CO₂H) C, H, N.

N-(4-(2-(4-(Cyclohexanecarboxamido)phenyl)-1H-imidazol-5-yl)-N-cyclohexylbenzamide: mp 250-2° C. ¹H NMR (DMSO-d₆, δ in ppm): 10.02 (s, 1 H), 8.18 (d, J=8.0 Hz, 1 H), 7.95 (d, J=8.8, Hz, 2 H), 7.94-7.89 (m, 3 H), 7.89 (d, J=8.4 Hz, 2 H), 7.74 (d, J=8.8 Hz, 2 H), 3.79-3.75 (m, 1 H), 2.36 (tt, J=8.4, 3.2 Hz, 1 H), 1.83-1.27 (m, 20 H). MS: [EI] m/e 471.4 [M+H]⁺. Anal: (C₂₉H₃₄N₄O₂-3.12 H₂O-CF₃CO₂H) C, H, N.

N-(4-(5-(4-(Cyclohexylcarbamoyl)phenyl)-1H-imidazol-2-yl)phenyl)cycloheptanecarboxamide: mp 240-2° C. ¹H NMR (DMSO-d₆, δ in ppm): 10.18 (s, 1 H), 8.28 (d, J=8.0 Hz, 1 H), 8.15 (s, 1 H), 8.05 (d, J=8.8, Hz, 2 H), 7.94 (br. s, 4 H), 7.86 (d, J=8.8 Hz, 2 H), 3.82-3.75 (m, 1 H), 2.58-2.49 (m, 1 H), 1.89-1.27 (m, 22 H). MS: [EI] m/e 485.4 [M+H]⁺. Anal: (C₃₀H₃₆N₄O₂-1.84 H₂O-0.33 CF₃CO₂H) C, H, N.

### Synthetic Scheme 13

4-Nitrobenzamidine HCl (21). (prepared by the known method Journal of Organic Chemistry 55, 7, 1990, 2005-2004) To a solution of 4-nitrobenzonitrile (21) (25.5 g, 172 mmol) in dry methanol (230 ml) was added a solution of sodium methoxide (1 g, 18.5 mmol) and the solution warmed until complete dissolution of the solid. The solution was stirred at room temperature for 55 h at which time solid NH₄Cl (9.5 g, 177 mmol) was added and the mixture heated at 45° C for 48 h. The mixture was cooled to room temperature and the resulting solid collected by filtration, rinsed with acetone and dried to give the product as a yellow solid (21.6 g, 107 mmol, 62%). The crude product was used as is in subsequent steps.

3-(2-(4-nitrophenyl)-1H-imidazol-5-yl)benzonitrile (134). To a refluxing solution of 4-nitrobenzamidine HCl (32) (930 mg, 4.5 mmol) and NaHCO₃ (4×, 1.5 g, 18 mmol) in THF (8 mL) and H₂O (2.5 mL) was added a solution of 3-(2-bromoacetyl)benzonitrile (133) (1 g, 4.5 mmol) in dry THF (2 mL) dropwise via syringe and the mixture heated at reflux for 1.5 h. The solvent was removed and the resulting residue sonicated in H₂O and the solid collected by filtration and dried to give 1.323 g of a black solid that was used as is.

3-(2-(4-nitrophenyl)-1H-imidazol-5-yl)benzoic acid (135). A solution 3-(2-(4-nitrophenyl)-1H-imidazol-5-yl)benzonitrile (134) (1.32 g, 4.6 mmol) in aqueous 20% KOH (40 mL) was heated at reflux for 1.5 h. The solution was cooled and adjusted to pH˜6 with 20% HCl and the resulting solid collected by filtration and dried to give 1.541 g of an orange solid that was used as is.

3-(2-(4-nitrophenyl)-1H-imidazol-5-yl)-N-(pyridin-2-yl)benzamide (136). To a suspension of 3-(2-(4-nitrophenyl)-1H-imidazol-5-yl)benzoic acid (135) (0.5 g, 1.62 mmol) in dry dichloromethane (10 mL) was added (COCl)₂ (1.5 eq, 0.31 g, 0.212 mL, 2.4 mmol) and the mixture warmed at 35° C. for 7 h. The solvent was removed under reduced pressure to give a solid residue. The residue was dissolved in dry pyridine (5 mL) and 2-aminopyridine (1.2eq, 183 mg, 1.95 mmol) was added as a solid and the mixture stirred for 15 h. The mixture was poured into H₂O and the resulting solid collected by filtration and dried to give 0.518 g of a brown orange solid that was used as is in the following step.

3-(2-(4-adamantylamidophenyl)-1H-imidazol-5-yl)-N-(pyridin-2-yl)benzamide (137). To a solution of 3-(2-(4-nitrophenyl)-1H-imidazol-5-yl)-N-(pyridin-2-yl)benzamide (136) (0.5 g, 1.3 mmol) in CH₃OH (25 mL) was added Raney Nickel and the mixture vacuum purged using H₂ and the mixture stirred under H₂ for 15 h. The solution was filtered through celite to remove the catalyst and the filtrate concentrated to give a solid residue.

The residue was dissolved in dry pyridine (10 mL) and 1-adamantane carbonyl chloride (1.5 eq, 270 mg, 1.35 mmol) added as a solid. The mixture was stirred at room temperature for 18 h and poured into H₂O and the solid collected by filtration. The resulting solid was collected by filtration and purified by HPLC (C18, ACN/TFA/H₂O) to give the product as a solid. (58 mg, 0.112 mmol, 8%) Mp: 205° C. ¹H NMR (400 MHz, DMSO-d₆) δ12.60 (apparent d, 1H), 10.78 (s, 1H), 9.25 (s, 1H), 8.49 (s, 1H), 8.23 (d, J=8.4 Hz, 1H), 8.07 (d, J=8 Hz, 1H), 7.95 (m, 2H), 7.88 (m, 3H), 7.78 (d, J=8.8 Hz,2H), 7.51 (t, J=7.6 Hz, 7.6 Hz, 1H), 7.18 (m, 1H), 2.03 (bs, 3H), 1.93 (m, 6H), 1.72 (bs, 6H) EIMS m/z M⁺¹ 518.4. Anal. (C, H, N)

### EXAMPLE 2

### Suppression of IgE Response

The inhibitory activity of the small molecules of the preferred embodiments were assayed using both the ex vivo and in vivo assays as described above. All of the compounds presented above were active in suppressing the IgE response. In the ex vivo assay, compounds in Genera 1-4 produced 50% inhibition at concentrations ranging from 1 pM to 100 μM. In the in vivo assay, the compounds were effective at concentrations ranging from less than about 0.01 mg/kg/day to about 100 mg/kg/day, when administered in divided doses (e.g., two to four times daily) for at least two to seven consecutive days. Thus, the small molecule inhibitors of the preferred embodiments are disclosed as being useful in lowering the antigen-induced increase in IgE concentration, and consequently, in the treatment of IgE-dependent processes such as allergies in general and allergic asthma in particular.

### EXAMPLE 3

### Effects on Cellular Proliferation

A variety of experiments were performed in an effort to determine the effect of the imidazole compounds on cellular proliferation. These experiments ultimately measured ³H-thymidine incorporation into proliferating cell DNA. The specific procedure varied with the cells and the stimuli. Cells derived from mouse spleen were cultured at 3 million per ml; cell lines were seeded at 0.1 to 1 million per ml. Splenic B cells were isolated by T cell depletion and stimulated with phorbol myristate acetate (PMA) (10 ng/ml) plus ionomycin (100 nM), or IL-4 (10 ng/ml) plus anti-CD40 Ab (100 ng/ml). T cells were depleted prior to culture by incubating spleen cells first with a cocktail of anti-Thy1 ascites (10%), anti-CD4 Ab (0.5 μg/ml) and anti-CD8 Ab (0.5 μg/ml), followed by guinea pig complement (adsorbed). Cell lines were unstimulated or stimulated with Human Epidermal Growth Factor (EGF) (100 ng/ml). All cells were cultured in 96-well plates for 2-3 days and pulsed for 6 to 14 hours with 50 μl of 3H-thymidine (50 μCi/ml).

In spleen cells, certain compounds of the preferred embodiments suppressed B cell proliferation responses to PMA/ionomycin and IL-4/anti-CD40 Ab with approximately the same potencies as it suppressed in vitro IgE responses to IL-4/anti-CD40 Ab. Similar inhibition potencies were obtained for certain compounds of the preferred embodiments in ConA-stimulated T cell proliferation and LPS-stimulated B cell proliferation (preformed by MDS Pharma), suggesting a lack of specificity in the action of these drugs. On the other hand, a battery of immunological tests performed with certain compounds of the preferred embodiments demonstrated little other effects other than inhibition of ConA-stimulated cytokine release.

In tumor cells, the results with splenic lymphocytes led to a further analysis of cellular proliferation by measuring the growth of tumor cells in the presence of these drugs. The initial analysis was performed with murine M12.4.1 lymphoma cells, either un-stimulated or stimulated with IL-4/anti-CD40 Ab. Certain compounds of the preferred embodiments suppressed the proliferation of M12.4.1 cells but with lower potency that observed in stimulated spleen cells. However, the potency of compounds of the preferred embodiments increased when the cells were cultured with IL-4/anti-CD40 Ab. This stimulation is known to induce the activity of NF-κB in M12.4.1 cells.

A similar approach was used to establish selectivity of the anti-proliferative activity by testing a battery of tumor lines derived from a variety of tissues, mostly human in origin. An attempt was made to generate proliferation data from at least 2 cell lines from each tissue selected. Only a handful of cell lines were inhibited by 100 nM or less of each compound while most the balance of the cells required much higher concentrations. Because of the known character of some of the tested cell lines and previous Western blot results with the compounds, there is evidence to suggest a link between NF-κB inhibition and the action of the drugs. Breast cancer cells offer a good model for testing this phenomenon because they are predominantly of 2 types; estrogen receptor (ER) -positive and ER-negative. The latter cells tend to be less differentiated, have a higher density of EGF receptor expression, and are more resilient to treatment. Proliferation of ER-negative/EGFR-positive cells also tends to be driven by NF-κB and thus a selection of these cells were tested for proliferation responses to drug in vitro. The proliferation of all of the EGF-responsive cell lines was potently inhibited by compounds of the preferred embodiments in vitro. Conversely, only 2 of the 5 ER-positive cell lines were potently inhibited by drug.

Certain compounds of the preferred embodiments exert an anti-proliferative activity to T and B lymphocytes exposed to a variety of immunogenic stimuli in vitro. These actions are highly potent and parallel their IgE-suppression activity. Although the mechanism of this action is unresolved, much is known about the mechanism of IL-4/anti-CD40 Ab-induced IgE production. A major factor in this response is the transcription activator, NF-κB. This factor has been implicated in the proliferation of a number of tumor cells and thus these drugs were tested for activity on the proliferation of various tumor cell lines in vitro. Our experiments revealed that a number of tumor cell lines are sensitive to the effects of compounds of the preferred embodiments, and that proliferation of many of the sensitive lines may be driven by NF-κB factors. However, other cell lines known to be driven by factors other than NF-κB (e.g., the ER-positive HCC 1500 and ZR-75-1). Thus, certain compounds of the preferred embodiments appears to selectively act on certain tumor cells. Other compounds disclosed in accordance with the present invention are-also expected to exhibit similar characteristics, particularly those compounds which are structurally similar to certain compounds of the preferred embodiments.

Treatment Regimens

The amount of the imidazole compounds which can be effective in treating a particular allergy or used as an anti-proliferation agent will depend on the nature of the disorder, and can be determined by standard clinical techniques. The precise dose to be employed in a given situation will also depend on the choice of compound and the seriousness of the condition, and should be decided according to the judgment of the practitioner and each patient's circumstances.

As an anti-allergy therapy, appropriate dosages can be determined and adjusted by the practitioner based on dose response relationships between the patient's IgE levels as well as standard indices of pulmonary and hemodynamic changes. Moreover, those skilled in the art will appreciate that dose ranges can be determined without undue experimentation by following the protocol(s) disclosed herein for ex vivo and in vivo screening (See for example Hasegawa et al., J. Med. Chem. 40: 395-407 (1997) and Ohmori et al., Int. J. Immunopharmacol. 15:573-579 (1993); employing similar ex vivo and in vivo assays for determining dose-response relationships for IgE suppression by naphthalene derivatives; incorporated herein by reference).

Initially, to exert anti-allergic or anti-asthmatic effects, suitable dosages of the compounds will generally range from about 0.001 mg to about 300 mg per kg body weight per day in divided doses, more preferably, between about 0.01 mg and 100 mg per kg body weight per day in divided doses. The compounds are preferably administered systemically as pharmaceutical formulations appropriate to such routes as oral, aerosol, intravenous, subcutaneously, or by any other route which may be effective in providing systemic dosing of the active compound. The compositions of pharmaceutical formulations are well known in the art. The treatment regimen preferably involves periodic administration. Moreover, long-term therapy may be indicated where allergic reactions appear to be triggered by continuous exposure to the allergen(s). Daily or twice daily administration has been effective in suppressing the IgE response to a single antigen challenge in animals when carried out continuously from a period of two to seven consecutive days. Thus, in a preferred embodiment, the compound is administered for at least two consecutive days at regular periodic intervals. However, the treatment regimen, including frequency of dosing and duration of treatment may be determined by the skilled practitioner, and modified as needed to provide optimal IgE down-regulation, depending on nature of the allergen, the dose, frequency, and duration of the allergen exposure, and the standard clinical indices.

In a preferred embodiment, an IgE-suppressing compound can be administered in conjunction with one or more of the other small molecule inhibitors disclosed, in order to produce optimal down-regulation of the patient's IgE response. Further, it is envisioned that one or more of the compounds of the preferred embodiments can be administered in combination with other drugs already known or later discovered for treatment of the underlying cause as well as the acute symptoms of allergy or asthma. Such combination therapies envisioned within the scope of embodiments include mixing of one or more of the small molecule IgE-inhibitors together with one or more additional ingredients, known to be effective in reducing at least one symptom of the disease condition. In a variation, the small molecule IgE-inhibitors herein disclosed can be administered separately from the additional drugs, but during the same course of the disease condition, wherein both the IgE-inhibitor(s) and the palliative compounds are administered in accordance with their independent effective treatment regimens.

As an anti-proliferative therapy, the appropriate dose of the imidazole compounds disclosed herein can be determined by one skilled in the art. Pharmacologists and oncologists can readily determine the appropriate dose required for each individual patient without undue experimentation, based upon standard treatment techniques used for other anti-proliferation and chemotherapeutic agents.

Initially, suitable dosages of the anti-proliferation imidazole compounds will generally range from about 0.001 mg to about 300 mg per kg body weight per day in divided doses, more preferably, between about 0.01 mg and 100 mg per kg body weight per day in divided doses. Most preferably, to exert anticancer effects, the dose will range from about 1 mg to 100 mg per kg body weight per day. The compounds are preferably administered systemically as pharmaceutical formulations appropriate to such routes as oral, aerosol, intravenous, subcutaneously, or by any other route which may be effective in providing systemic dosing of the active compound.

Ideally one or more imidazole compounds of the preferred embodiments should be administered to achieve peak plasma concentrations of the active agent, as determined by one of skill in the art. To achieve adequate plasma levels, the pharmaceutical formulation can be injected intravenously in an appropriate solution, such as a saline solution or administered as a bolus of the active ingredient.

The treatment regimen used in accordance with preferred embodiments preferably involves periodic administration. Moreover, as with other chemotherapeutic agents, long-term therapy may be indicated. Weekly, daily or twice daily administration for a period of one to three years may be required for some patients. Thus, in a preferred embodiment, the compound is administered for at least six months at regular periodic intervals. However, the treatment regimen, including frequency of dosing and duration of treatment may be determined by the skilled practitioner, and modified as needed to provide optimal anti-proliferation effects, depending on nature of the disease, the extent of abnormal cell growth, the type of cancer, the tissues affected, and standard clinical indices.

One skilled in the art will understand that the ideal concentration of the anti-proliferation compounds in the formulation depends upon several pharmacokinetic parameters, such as, absorption, inactivation, metabolism and clearance rates of the drug as well as other known factors. One skilled in the art will also appreciate that the concentration will vary with the severity of the condition to be treated. Other factors which may affect the treatment dose include, tumor location, age and gender of the patient, other illnesses, prior exposure to other drugs, and the like. One skilled in the art will appreciate that for any particular patient, specific treatment regimens will be evaluated and adjusted over time according to the individual patient's requirements and according to the professional judgment of the medical practitioner administering the treatment.

In one preferred embodiment, compounds are orally administered. Preferably, oral formulations will include inert diluents or edible carriers. Oral dosages may be encapsulated in gelatin or formed into tablets. Oral administration may also be accomplished by using granules, grains or powders, syrups, suspensions, or solutions. One skilled in the art will understand that many acceptable oral compositions may be used in accordance with preferred embodiments. For example, the active compound may be combined with standard excipients, adjuvants, lubricants, sweetening agents, enteric coatings, buffers, stabilizing agents and the like.

In another embodiment, the active compound may be modified to include a targeting moiety that targets or concentrates the compound at the active site. Targeting moieties include, but are not limited to, antibodies, antibody fragments or derivatives, cytokines, and receptor ligands expressed on the cells to be treated.

In preferred embodiments, compounds are administered in conjunction with other active agents, which either supplement or facilitate the action of the imidazole compound or cause other independent ameliorative effects. These additional active agents include, but are not limited to, antifungals, antivirals, antibiotics, anti-inflammatories, and anticancer agents. Protectants, which include carriers or agents which protect the active imidazole compound from rapid metabolism, degradation or elimination may also be used. Controlled release formulations can also be used in accordance with preferred embodiments.

In another embodiment, one or more anti-proliferation compounds may be administered in conjunction with one or more other anti-cancer agents or treatments to produce optimal anti-proliferative effects. Anti-cancer agents include, but are not limited to, alkylating agents (lomustine, carmustine, streptozocin, mechlorethamine, melphalan, uracil nitrogen mustard, chlorambucil cyclophosphamide, iphosphamide, cisplatin, carboplatin mitomycin thiotepa dacarbazine procarbazine, hexamethyl melamine, triethylene melamine, busulfan, pipobroman, and mitotane); antimetabolites (methotrexate, trimetrexate pentostatin, cytarabine, ara-CMP, fludarabine phosphate, hydroxyurea, fluorouracil, floxuridine, chlorodeoxyadenosine, gemcitabine, thioguanine, and 6-mercaptopurine); DNA cutters (bleomycin); topoisomerase I poisons (topotecan irinotecan and camptothecin); topoisomerase II poisons (daunorubicin, doxorubicin, idarubicin, mitoxantrone, teniposide, and etoposide); DNA binders (dactinomycin, and mithramycin); and spindle poisons (vinblastine, vincristine, navelbine, paclitaxel, and docetaxel).

Further, it is envisioned that one or more of the compounds of the preferred embodiments can be administered in combination with other therapies, such as radiation, immunotherapy, gene therapy and/or surgery, in order to treat hyperproliferative diseases, including cancer. Such combination therapies envisioned within the scope of embodiments include mixing of one or more of the imidazole compounds together with one or more additional ingredients, known to be effective in reducing at least one symptom of the disease condition. In a variation, the imidazole compounds herein disclosed may be administered separately from the additional drugs, but during the same course of the disease condition, wherein both the imidazole compound and the palliative compounds are administered in accordance with their independent effective treatment regimens.

While a number of preferred embodiments and variations thereof have been described in detail, other modifications and methods of use will be readily apparent to those of skill in the art. Accordingly, it should be understood that various applications, modifications and substitutions may be made of equivalents without departing from the spirit of the invention or the scope of the claims.

What is claimed is:

1. A pharmaceutical composition for treating or preventing an allergic reaction associated with increased IgE levels or inhibiting cellular proliferation in a mammal comprising any one or more of the following compounds: 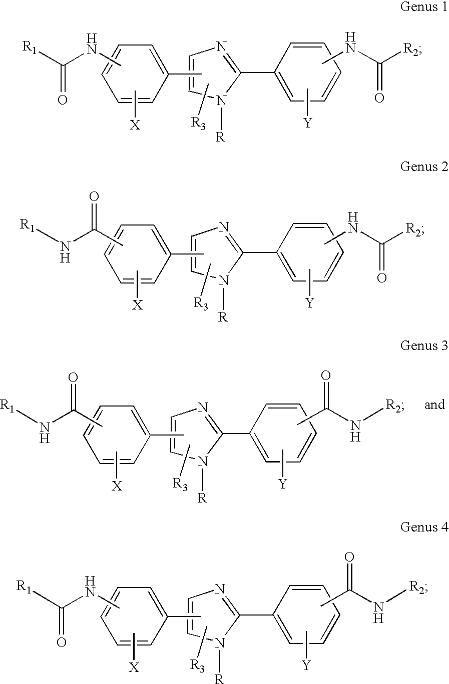
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, diaikylarninoalkyl, hydroxyalkyl, OH, OCH₃, COOH, COOR′ COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl.

2. The compound of claim 1, wherein said polycyclic aliphatic group is selected from the group consisting of adamantyl, bicycloheptyl, camphoryl, bicyclo[2,2,2]octanyl, and norbomyl.

3. The compound of claim 1, wherein said heterocyclic and said substituted heterocyclic is selected from the group consisting of pyridines, thiazoles, isothiazoles, oxazoles, pyrimidines, pyrazines, furans, thiophenes, isoxazoles, pyrroles, pyridazines, 1,2,3-triazines, 1,2,4-triazines, 1,3,5-triazines, pyrazoles, imidazoles, indoles, quinolines, iso-quinolines, benzothiophines, benzofurans, parathiazines, pyrans, chromenes, pyrrolidines, pyrazolidines, imidazolidines, morpholines, thiomorpholines, and the corresponding saturated heterocyclics.

4. The pharmaceutical composition of claim 1, further comprising at least one additional ingredient which is active in reducing at least one symptom associated with said allergic reaction, cell proliferation and/or inhibition of cytokines or leukocytes.

5. A method for treating or preventing an allergic reaction and/or for inhibiting cytokines or leukocytes in a mammal wherein said reaction is caused by an increase in IgE levels comprising administering an IgE-suppressing amount of at least one compound of claim 1.

6. The method of claim 5 further comprising administering at least one additional ingredient which is active in reducing at least one symptom associated with said allergic reaction.

7. The method of claim 6, wherein said at least one additional ingredient is selected from the group consisting of a short-acting β₂-adrenergic agonist, a long-acting β₂-adrenergic agonist, an antihistamine, a phosphodiesterase inhibitor, an anticholinergic agent, a corticosteroid, an inflammatory mediator release inhibitor and a leukotriene receptor antagonist.

8. The method of claim 6, wherein said at least one additional ingredient is combined with said at least one IgE-suppressing compound in a pharmaceutically acceptable diluent and co-administered to the mammal.

9. The method of claim 8, wherein said at least one IgE-suppressing compound is administered at a dose of about 0.01 mg to about 100 mg per kg body weight per day.

10. The method of claim 9, wherein said dose is administered in divided doses at regular periodic intervals.

11. The method of claim 10, wherein said regular periodic intervals occur daily.

12. A method for treating or preventing asthma in a mammal comprising administering an IgE-suppressing amount of at least one compound of claim 1.

13. The method of claim 12 further comprising administering at least one additional ingredient which is active in reducing at least one symptom associated with said asthma.

14. The method of claim 13, wherein said additional ingredient is selected from the group consisting of a short-acting β₂-adrenergic agonist, a long-acting β₂-adrenergic agonist, an antihistamine, a phosphodiesterase inhibitor, an anticholinergic agent, a corticosteroid, an inflammatory mediator release inhibitor and a leukotriene receptor antagonist.

15. A method for inhibiting cellular proliferation in a mammal comprising administering an amount of at least one compound of claim 1.

16. The method of claim 15 further comprising administering at least one additional ingredient which is active in reducing at least one symptom associated with said cellular proliferation.

17. The method of claim 16, wherein said at least one additional ingredient is selected from the group consisting of antifungals, antivirals, antibiotics, anti-inflammatories, and anticancer agents.

18. The method of claim 16, wherein said at least one additional ingredient is selected from the group consisting of alkylating agent, antimetabolite, DNA cutter, topoisomerase I poison, topoisomerase II poison, DNA binder, and spindle poison.

19. The method of claim 16, wherein said at least one additional ingredient is combined with said at least one compound of claim 1 in a pharmaceutically acceptable diluent and co-administered to the mammal.

20. The method of claim 19, wherein said at least one compound of claim 1 is administered at a dose of about 0.01 mg to about 100 mg per kg body weight per day.

21. The method of claim 20, wherein said dose is administered in divided doses at regular periodic intervals.

22. The method of claim 21, wherein said regular periodic intervals occur daily.

23. The method of claim 15 further comprising administering at least one other therapy which is effective in ameliorating at least one symptom associated with cellular hyperproliferation.

24. The method of claim 23, wherein said therapy is an anti-cancer therapy.

25. The method of claim 23, wherein said therapy is selected from the group consisting of radiation, immunotherapy, gene therapy, and surgery.

26. The pharmaceutical composition of claim 1, wherein R₁ and R₂ are independently selected from Genera 1-4, preferred substituents for R₁ and R₂ are selected from the following:

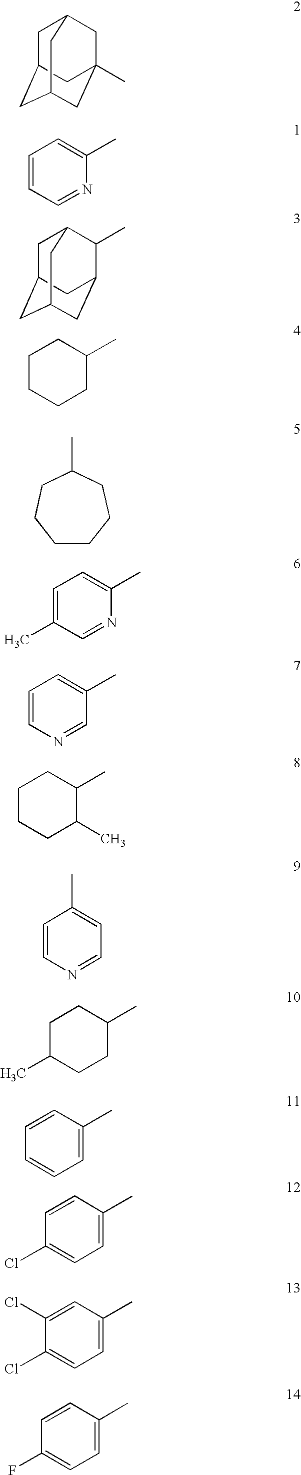

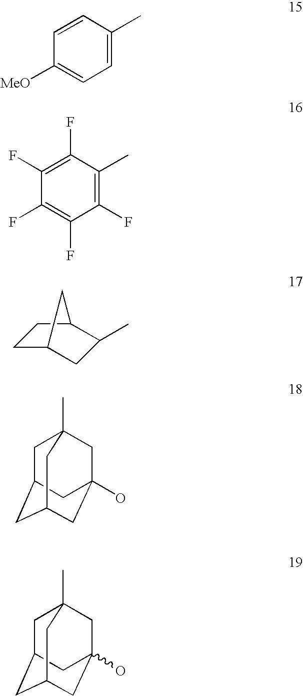

27. A method of preparing a compound or salt thereof having the formula: 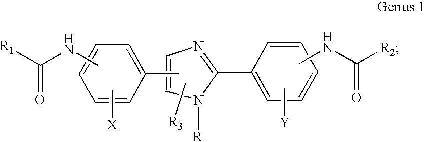
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF_{3,} OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises steps:
converting a Y-substituted-nitro-benzonitrile to a Y-substituted nitro-benzamidine;
reacting the Y-substituted nitro-benzamidine with X-substituted nitro-phenacyl halide to form a species of the formula 13 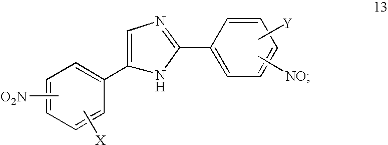
reducing the species of the formula 13 to form a species of the formula 14 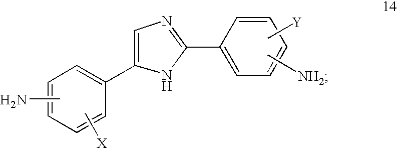
and
acylating the species of the formula 14 to form a species of the formula 15

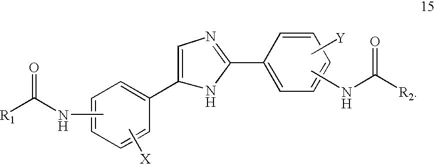

28. A method of preparing a compound or salt thereof having the formula: 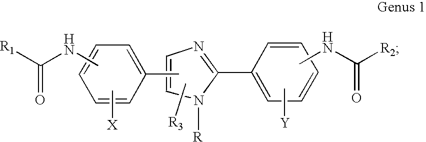
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises steps:
converting a Y-substituted nitro-benzonitrile to a Y-substituted nitro-benzamidine; benzamidine;
reacting the Y-substituted nitro-benzamidine with X-substituted acetamido-phenacyl halide to form species of the formula 74 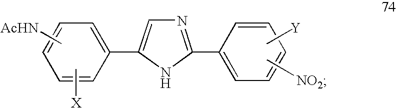
hydrolyzing the species of the formula 74 to form a species pf the formula 75 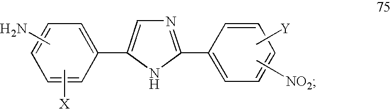
acylating the species of the formula 75 to from a species of the formula 76 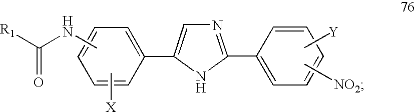
reducing the species of the formula 76 to form a species of the formula 77 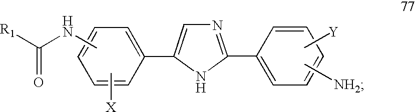
and
acylating the species of the formula 77 to from a species of the formula 78

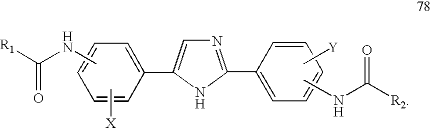

29. A method of preparing a compound or salt thereof having the formula: 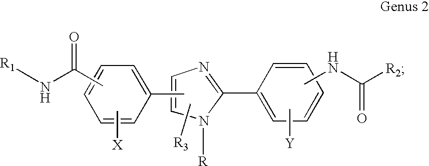
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises the following steps:
converting a Y-substituted-nitro-benzonitrile to a Y-substituted nitro-benzamidine;
reacting the Y-substituted nitro-benzamidine with X-substituted cyano-phenacyl halide to form a species of the formula 92 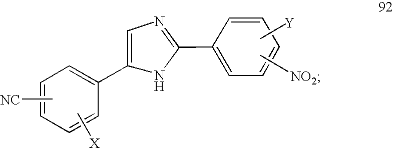
reducing the species of the formula 92 to form a species of the formula 93 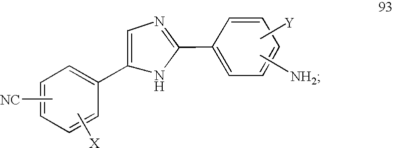
acylating the species of the formula 93 and subsequently performing a hydrolysis to form a species of the formula 94 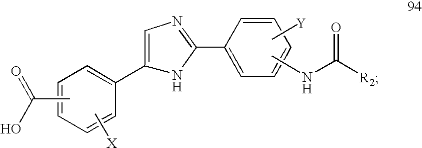
and
aminating the species of the formula 94 to form a species of the formula 95

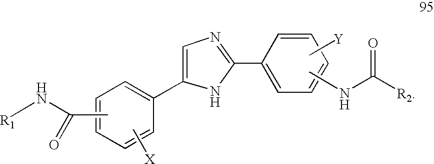

30. A method of preparing a compound or salt thereof having the formula: 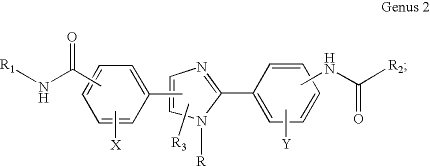
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises the following steps:
converting a Y-substituted nitro-benzonitrile to a Y-substituted nitro-benzamidine;
converting methyl X-substituted 4-acetyl benzoate to a methyl X-substituted 4-(alpha-bromoacetyl) benzoate;
reacting the Y-substituted nitro-benzamidine with methyl X-substituted 4-(alpha-bromoacetyl)benzoate to form species of the formula 103 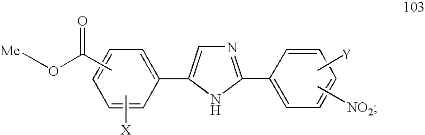
hydrolyzing the species of the formula 103 to form a species of the formula 104 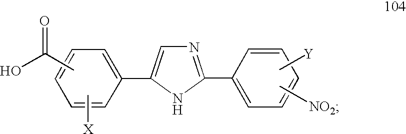
aminating the species of the following formula 104 to form a species of the formula 105 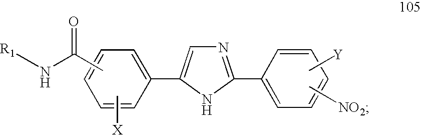
and
reducing and amidating the formula 105 to form a species of the formula 106

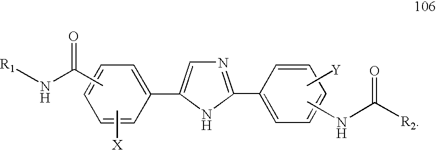

31. A method of preparing a compound or salt thereof having the formula: 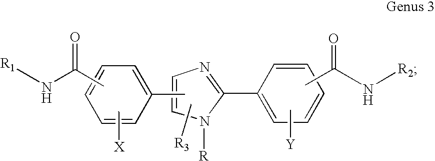
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises the following steps:
converting a Y-substituted-alkoxycarbonyl-benzonitrile to a Y-substituted alkoxycarbonyl-benzamidine;
reacting the Y-substituted alkoxycarbonyl-benzamidine with X-substituted cyano-phenacyl halide to form a species of the formula 142 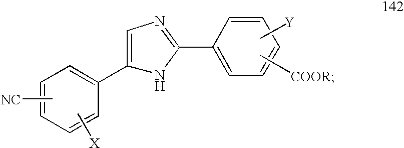
hydrolyzing the species of the formula 142 to form a species of the formula 143 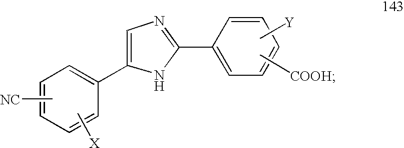
amidating the species of the formula 143 to form a species of the formula 143a 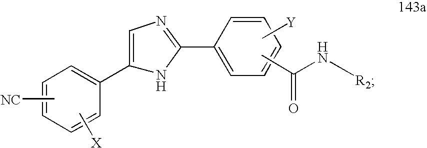
hydrolyzing the species of the formula 143a to form a species of the formula 143b 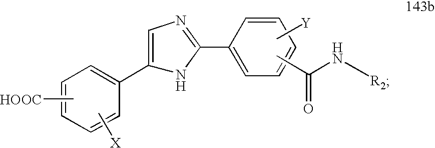
and
amidating the species of the formula 143b to form a species of the formula 144

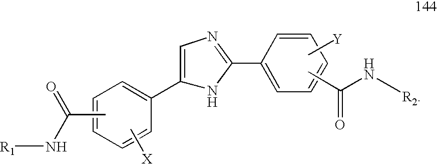

32. A method of preparing a compound or salt thereof having the formula: 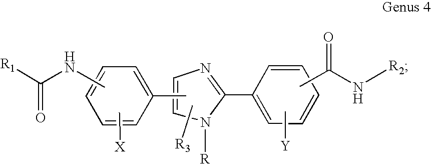
wherein R is selected from the group consisting of H, C₁-C₅ alkyl, benzyl, p-fluorobenzyl, and dialkylaminoalkyl, wherein said C₁-C₅ alkyl is selected from the group consisting of a straight chain, branched or cyclic alkyl;
wherein R₃, X, and Y are independently selected from the group consisting of H, halogen, alkoxy, substituted alkoxy, alkyl, substituted alkyl, dialkylaminoalkyl, hydroxyalkyl, OH, OCH₃, COOH, CN, CF₃, OCF₃, NO₂, COOR″, CHO, and COR″;
wherein R₁ and R₂ are independently selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heterocyclic, and substituted heterocyclic, wherein said heterocyclic and said substituted heterocyclic contain 1-3 heteroatoms, wherein said heteroatom is independently selected from the group consisting of nitrogen, oxygen and sulfur;
wherein said substituents are selected from the group consisting of H, halogen, alkoxy, substituted alkoxy, alkyl, substituted alkyl, dialkylaminoalkyl, hydroxyalkyl, OH, OCH₃, COOH, COOR′COR′, CN, CF₃, OCF₃, NO₂, NR′R′, NHCOR′ and CONR′R′;
wherein R′ is selected from the group consisting of H, alkyl, substituted alkyl, C₃-C₉ cycloalkyl, substituted C₃-C₉ cycloalkyl, polycyclic aliphatic groups, phenyl, substituted phenyl, naphthyl, substituted naphthyl, heteroaryl and substituted heteroaryl, wherein said heteroaryl and said substituted heteroaryl contain 1-3 heteroatoms, wherein said heteroatom is independently selected from the group consisting of nitrogen, oxygen and sulfur; and
wherein R″ is selected from the group consisting of C₁-C₉ alkyl, wherein said C₁-C₉ alkyl is selected from the group consisting of straight chain alkyl, branched alkyl, and cyclic alkyl;
wherein said method comprises the following steps:
converting a Y-substituted-alkoxycarbonyl-benzonitrile to a Y-substituted alkoxycarbonyl-benzamidine;
reacting the Y-substituted alkoxycarbonyl-benzamidine with X-substituted nitro-phenacyl halide to form a species of the formula 152 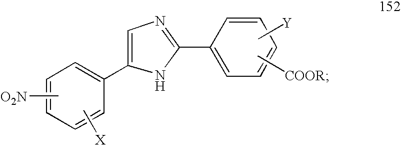
reducing the species of the formula 152 to form a species of the formula 153 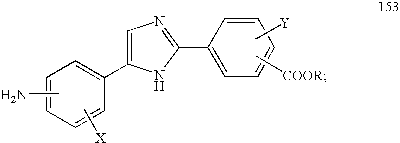
acylating the species of the formula 153 to form a species of the formula 154 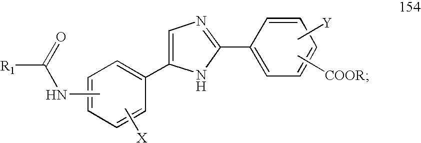
and
anidating the species of the formula 154 to form a species of the formula 155 [chemical structure]
